(19) United States
(12) Patent Application Publication
(10) Pub. No.: US 2011/0229513 A1
(43) Pub. Date: Sep. 22, 2011

(54) LPS Based Vaccines

(76) Inventors: Andrew D. Cox, Ottawa (CA); Frank St. Michael, Ottawa (CA); James C. Richards, Ottawa (CA); Richard E. Moxon, Oxfordshire (GB)

(21) Appl. No.: 13/062,299

(22) PCT Filed: Sep. 8, 2009
(86) PCT No.: PCT/CA09/01201

§ 371 Date: May 16, 2011

(30) Foreign Application Priority Data
Sep. 5, 2008 (US) 61094495

(51) Int. Cl. A61K 39/385; C07K 14/00; C07K 14/765; C07K 14/34; C07K 14/33; C07K 1/113; C12P 21/00; A61P 37/04; A61P 31/04

(52) U.S. Cl. 424/197.11; 530/359; 530/363; 435/68.1; 530/395

(57) ABSTRACT

The removal of the glycosidic phosphate from the reducing end of the derived LPS molecule creates an aldehydo functionality which causes the formation of an immunologically dominant neo-epitope. Conjugation to the reducing end of a carbohydrate molecule following removal of the glycosidic phosphate traps the reducing glucosamine residue in an open-chain form which surprisingly was found to dominate the immune response. We therefore modified our conjugation strategy to avoid this open-chain form, by utilising the amino functionality created by the isolated amidase activity from Dictyostelium discoideum, concomitant with a unique blocking and un-blocking strategy to protect the immunologically important phosphoethanolamine inner core residue. These antigenic structures are useful in producing vaccines and compounds helpful in combating Gram-negative bacteria. Also described are specific structures of the carbohydrate molecules derived from a variety of Gram-negative bacteria, which when presented appropriately as a glycoconjugate will facilitate a functional immune response to the target core oligosaccharide region.

### PRIOR APPLICATION INFORMATION

This application claims the benefit of U.S. Provisional Patent Application 61/094,495, filed Sep. 5, 2008.

### BACKGROUND OF THE INVENTION

An effective broadly cross-reactive vaccine to combat disease caused by the serogroup B meningococcus remains the Holy Grail for researchers in this field. Glycoconjugate vaccines based on the capsular polysaccharides are currently available and have proven successful in protecting against serogroups A, C, W-135 and Y. Unfortunately the serogroup B capsule consists of an α-2,8-sialic acid polymer which is poorly immunogenic, especially in infants as it mimics glyco-modifications on host neuronal cells. Therefore alternative vaccine antigens are being sought including modified capsular polysaccharide, outer membrane vesicles, attenuated vaccines, common antigens identified in Neisseria lactamica and outer membrane proteins identified from genomic and signature tagged mutagenesis approaches. Some of these candidates are in early phase I or phase II trials: N. lactamica OMV, PorA and a genome derived pentavalent vaccine. Our strategy is to use inner core LPS that has been shown to be conserved in the majority of NmB strains, accessible to antibodies and able to elicit functional Abs against NmB strains.

Gram-negative bacteria can cause diseases of significant public health and economic concern in humans and other animals. Vaccine strategies are being pursued to combat these infections. These strategies are based on the identification of conserved, immunogenic cell surface components; however, the detection of conserved molecules that would confer protection against the vast majority of strains from a single species has proven problematic.

The outer leaflet of the outer membrane of all Gram-negative bacteria contains an amphiphillic carbohydrate molecule termed lipopolysaccharide (LPS). The LPS of most such bacteria consist of an oligosaccharide group attached to a Lipid A moiety, and can be represented by a general formula, shown in FIG. 1, where each R represents a fatty acid group that may be further acylated. While the R groups differ between species, they are generally associated with holding the LPS in a cell wall and with toxic effects.

The lipid A region is responsible for the endotoxic activity of the Gram-negative bacterium and consists in most species of a disaccharide of glucosamine sugars that are phosphorylated and contain both ester and amide linked fatty acids. It is generally conserved, and typically has the phosphorylation pattern shown in FIG. 1.

The oligosaccharide portion of this structure is highly variable. An O-antigenic polymeric repeating unit (O-antigen) can be present or absent beyond the core oligosaccharide, nearest the Lipid A portion of the LPS molecule. The core oligosaccharide can be arbitrarily divided into an outer and inner core and is connected to the lipid A region via one or more ketose sugar(s), 2-keto-3-deoxy-octulosonic acid (Kdo). The O-antigen is a variable moiety between strains of the same species and is often the antigen responsible for the serotyping schemes adopted to classify a species. Due to its variable nature within most species the O-antigen is not a good vaccine candidate as antibodies directed to one O-antigen will be serotype specific, and not offer protection to other serotypes of the same strain. Similarly the outer core region can be somewhat variable within a species and is also therefore not a good vaccine candidate. However what is arbitrarily termed the inner core oligosaccharide has been found to be conserved within several species, and is the vaccine antigen of choice in this application. However the technology described here would be equally applicable to the other LPS carbohydrate antigens, outer core oligosaccharide and O-antigens provided the complete LPS molecule is present.

The endotoxicity of the lipid A region is due to the fatty acid residues. Removal of the ester-linked fatty acids leaves an O-deacylated LPS species that is no longer endotoxic. Removal of all fatty acids i.e. both the amide and ester-linked fatty acids can be performed chemically, but involves harsh conditions which can effect other regions of the LPS molecule. Conserved regions of LPS molecules have been identified in the core oligosaccharide of several species.

LPS based vaccines generally require the removal of sufficient fatty acids from the lipid A region of the molecule to preclude endotoxicity and to derive a molecule that is amenable to conjugation strategies. Preferably, this de-toxification step does not modify the immunologically important carbohydrate epitopes on the LPS molecule, as these are expected to provide species-specific immunogenicity. Also, for reasons described herein, the detoxification step can be used to create functional groups that will facilitate conjugation strategies.

Current strategies used in the art to prepare LPS-based glycoconjugate vaccines, link the carbohydrate to a carrier protein either via the Kdo residues of O-deacylated LPS or of core oligosaccharides or via the derived lipid A region of the molecule. We have shown previously that conjugation via the Kdo residues does not optimally present the target core oligosaccharide region to the host's immune system and the resulting sera are not functional.

### SUMMARY OF THE INVENTION

According to an aspect of the invention, there is provided an immunogenic conjugate for eliciting a specific immune response to a Gram negative bacterium having a lipopolysaccharide (LPS) endotoxin, said conjugate having the formula I:

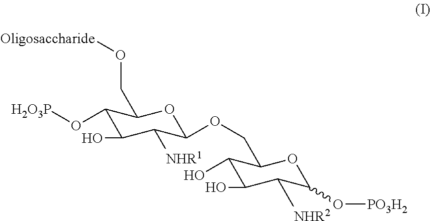

wherein one of R¹ and R² is a linker that is attached to a carrier protein, and the other of R¹ and R² is H or a C1-C20 acyl group;

‘Oligosaccharide’ represents at least five saccharide rings wherein each oligosaccharide comprises a saccharide having a general formula of:

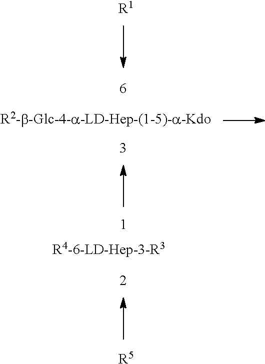

wherein R1 is H or α-D-glucose;
R² is H, β-D-glucose, β-D-galactose or a disaccharide of β-N-acetyl-D-glucosamine linked to the 3-position of a β-D-galactose, α-DD-heptose or α-LD-heptose;
R3 is H, phosphoethanolamine or α-D-glucose;
R4 is H or phosphoethanolamine; and
R5 is α-N-acetyl-D-glucosamine, α-LD-heptose or a disaccharide of or β-D-Glc-2-α-LD-Hep or a trisaccharide of β-D-Gal-(1-4)-β-D-Glc-2-α-LD-Hep or a tetrasaccharide of α-D-Gal-(1-4)-β-D-Gal-(1-4)-β-D-Glc-2-α-LD-Hep or a pentasaccharide of β-D-GalNAc-(1-3)-α-D-Gal-(1-4)-β-D-Gal-(1-4)-β-D-Glc-2-α-LD-Hep.
   and wherein the conjugate retains each phosphate and each phosphoethanolamine present in the corresponding portions of the natural LPS of the Gram negative bacterium;
   or a pharmaceutically acceptable salt thereof.

According to a further aspect of the invention, there is provided an immunogenic conjugate for eliciting a specific immune response to a Gram negative bacterium having a lipopolysaccharide (LPS) endotoxin, said conjugate having the formula I:

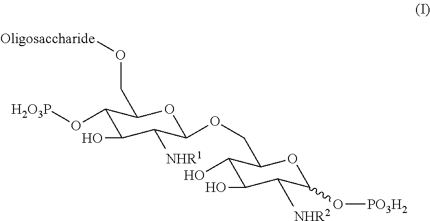

wherein one of R¹ and R² is a linker that is attached to a carrier protein, and the other of R¹ and R² is H or a C1-C20 acyl group;

‘Oligosaccharide’ represents at least five saccharide rings that comprise the corresponding saccharide rings of the lipopolysaccharide endotoxin of the Gram negative bacterium,

and wherein the conjugate retains each phosphate and each phosphoethanolamine present in the corresponding portions of the natural LPS of the Gram negative bacterium;

or a pharmaceutically acceptable salt thereof.

According to another aspect of the invention, there is provided a compound of Formula III:

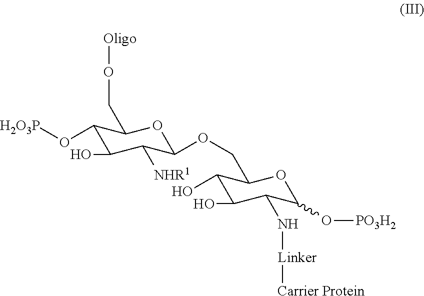

wherein said carrier protein is selected from the group consisting of CRM₁₉₇, tetanus toxoid (TT), human serum albumin (HSA), keyhole limpet hemocyanin (KLH), polydextran and MAP-4 peptide;

Oligo represents an oligosaccharide containing at least five contiguous saccharide rings of a moiety selected from the group consisting of

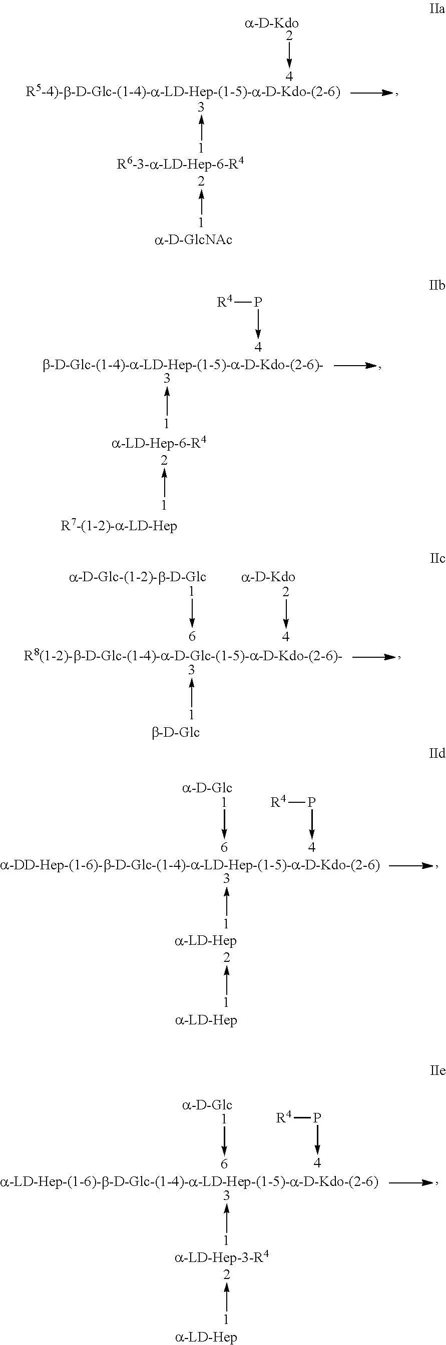

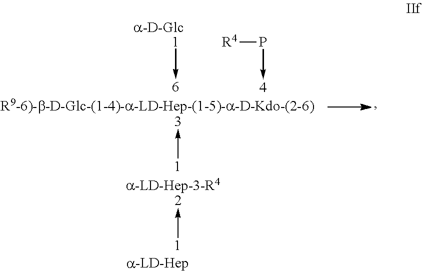

R¹ is H or an acyl group;

R⁴ is independently at each occurrence H or PEtn;

PEtn is phosphoethanolamine, P . . . PEtn is ethanolamine pyrophosphate, and PCho is phosphorylcholine;

R⁵ is H, beta-D-glucose, beta-D-galactose or disaccharide of beta-N-acetyl-D-glucosamine;

R⁶ is H, phosphoethanolamine (PEtn) or alpha-D-glucose;

R⁷ is selected from the group consisting of H, beta-D-Glc, a disaccharide of beta-D-Gal-(1-4)-beta-D-Glc, a trisaccharide of alpha-D-Gal-(1-4)-beta-D-Gal-(1-4)-beta-D-Glc and a tetrasaccharide of beta-D-GalNAc-(1-3)-alpha-D-Gal-(1-4)-beta-D-Gal-(1-4)-beta-D-Glc;

R⁸ is H, alpha-N-acetyl-D-glucosamine or alpha-D-glucose; and

R⁹ is H or alpha-LD heptose; and

said Linker is selected from the group consisting of a bond between the nitrogen of the glucosamine portion of the LPS and a carbon atom of the carrier protein, (C═O)C1-C8 alkylene, (C═O)C1-C8 alkenylene, (C═O)C1-C8 alkynylene, C1-C8 alkylene(C═O), C1-C8 alkenylene(C═O), C1-C8 alkynylene(C═O), (C═O)C1-C8 heteroalkylene, (C═O)C1-C8 heteroalkenylene, (C═O)C1-C8 heteroalkynylene, a peptide linker, C2-C20 polyethylene glycol),

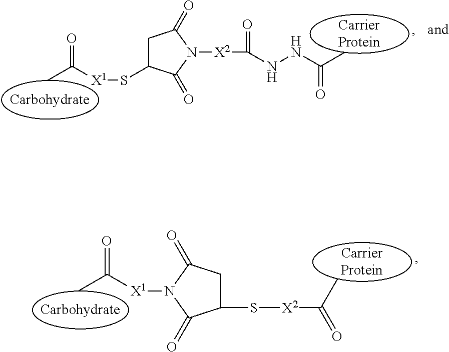

wherein the linker is the group connecting the carbohydrate and the carrier protein portions of the conjugate; and

X¹ and X² are each independently selected from the group consisting of C1-C8 alkylene, C1-C8 alkenylene, C1-C8 alkynylene, C1-C8 heteroalkylene, C1-C8 heteroalkenylene and C1-C8 heteroalkynylene;

or a pharmaceutically acceptable salt thereof.

According to a further aspect of the invention, there is provided an immunogenic conjugate for eliciting a specific immune response to a Gram negative bacterium having a lipopolysaccharide (LPS) endotoxin, said conjugate having at least 5 molecules of the carbohydrate depicted as

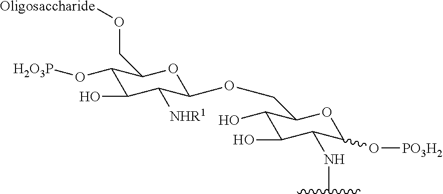

or a pharmaceutically acceptable salt thereof conjugated to a single carrier protein via a linker;

wherein one of R¹ and R² is a linker that is attached to a carrier protein, and the other of R¹ and R² is H or a C1-C20 acyl group;

‘Oligosaccharide’ represents at least five saccharide rings that comprise the corresponding saccharide rings of the lipopolysaccharide endotoxin of the Gram negative bacterium,

and wherein the conjugate retains each phosphate and each phosphoethanolamine present in the corresponding portions of the natural LPS of the Gram negative bacterium;

or a pharmaceutically acceptable salt thereof.

According to a further aspect of the invention, there is provided an immunogenic conjugate for eliciting a specific immune response to a Gram negative bacterium having a lipopolysaccharide (LPS) endotoxin, said conjugate depicted as

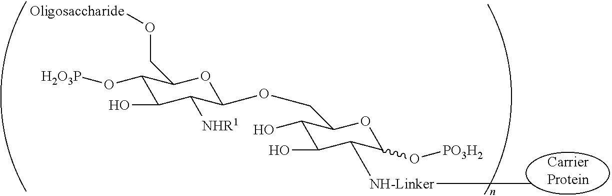

or a pharmaceutically acceptable salt thereof, wherein n is at least 5;

wherein one of R¹ and R² is a linker that is attached to a carrier protein, and the other of R¹ and R² is H or a C1-C20 acyl group;

‘Oligosaccharide’ represents at least five saccharide rings that comprise the corresponding saccharide rings of the lipopolysaccharide endotoxin of the Gram negative bacterium,

and wherein the conjugate retains each phosphate and each phosphoethanolamine present in the corresponding portions of the natural LPS of the Gram negative bacterium;

or a pharmaceutically acceptable salt thereof.

According to another aspect of the invention, there is provided a method to elicit a specific immune response to a Gram negative bacterium, comprising administering to a subject an effective amount of the conjugate described above or a vaccine composition comprising the conjugate described above.

According to a further aspect of the invention, there is provided a method to make an LPS-based immunological conjugate that induces an immune response effective against a Gram negative bacterium, comprising the steps of: obtaining a lipopolysaccharide (LPS) from the Gram negative bacterium; removing acyl groups linked to oxygen on the di-glucosamine of the reducing end portion of the LPS; removing at least one acyl group linked to N of the di-glucosamine reducing end of the LPS to provide an amine group; protecting any phosphoethanolamine groups attached to the oligosaccharide portion of the LPS: attaching a first end of a linking group to an amine group on the di-glucosamine; attaching a second end of the linking group to a carrier moiety; and de-protecting where necessary the protected phosphoethanolamine groups.

According to a further aspect of the invention, there is provided an LPS-based immunological conjugate prepared by the method described above.

According to another aspect of the invention, there is provided a method to elicit a specific immune response to a Gram negative bacterium, comprising administering to a subject an effective amount of the conjugate described above or a vaccine composition comprising the conjugate.

### BRIEF DESCRIPTION OF THE DRAWINGS

FIG. 1. Diagram showing general formula of LPS, where each R represents a fatty acid group that may be further acylated. While the R groups differ between species, they are generally associated with holding the LPS in a cell wall and with toxic effects.

FIG. 2. General formula of conjugate, wherein one of R¹ and R² is a linker that is attached to a carrier protein, and the other of R₁ and R² is H or a C1-C20 acyl group.

FIG. 3. a) Diagram showing generalised structures of possible oligosaccharides to be conjugated via the modified lipid A region to the carrier protein. For Neisseria meningitidis, R1 is H, R² is H, β-D-glucose, β-D-galactose or a disaccharide of β-N-acetyl-D-glucosamine linked to the 3-position of a β-D-galactose, R3 is H, phosphoethanolamine or α-D-glucose, R4 is H or phosphoethanolamine and R5 is α-N-acetyl-D-glucosamine. For Haemophilus influenzae, R1 is H, R2 is H, R3 is H, R4 is phosphoethanolamine and R5 is α-LD-heptose or a disaccharide of or β-D-Glc-2-α-LD-Hep or a trisaccharide of β-D-Gal-(1-4)-β-D-Glc-2-α-LD-Hep or a tetrasaccharide of α-D-Gal-(1-4)-β-D-Gal-(1-4)-β-D-Glc-2-α-LD-Hep or a pentasaccharide of β-D-GalNAc-(1-3)-α-D-Gal-(1-4)-β-D-Gal-(1-4)-O-D-Glc-2-α-LD-Hep. For Mannheimia haemolytica, R1 is α-D-glucose, R2 is α-DD-heptose, R3 is H, R4 is H and R5 is α-LD-heptose. For Actinobacillus pleuropneumoniae, R1 is α-D-glucose, R2 is α-DD-heptose, R3 is H, R4 is H and R5 is α-LD-heptose. For Pasteurella multocida, R1 is α-D-glucose, R2 is H or α-LD-heptose, R3 is H or phosphoethanolamine, R4 is H and R5 is α-LD-heptose. b) Diagram showing generalised structures of possible oligosaccharides to be conjugated for Moraxella catarrhalis, where R is H or α-N-acetyl-D-glucosamine.

FIG. 4. Diagram showing basal structure of carbohydrate molecule for Neisseria meningitidis applications, where R is H or a fatty acid, R¹ is H, phosphoethanolamine or α-D-glucose, R² is H or phosphoethanolamine and R³ is H, O-D-glucose, β-D-galactose or a disaccharide of 3-N-acetyl-D-glucosamine linked to the 3-position of a β-D-galactose.

FIG. 5. Diagram showing basal structure of carbohydrate molecule for Haemophilus influenzae applications, where R is H or a fatty acid, R¹ is H or phosphoethanolamine, R² is H or phosphoethanolamine and R³ is H or β-D-Glc or a disaccharide of β-D-Gal-(1-4)-β-D-Glc or a trisaccharide of α-D-Gal-(1-4)-β-D-Gal-(1-4)-β-D-Glc or a tetrasaccharide of β-D-GalNAc-(1-3)-α-D-Gal-(1-4)-β-D-Gal-(1-4)-O-D-Glc.

FIG. 6. Diagram showing basal structure of carbohydrate molecule for Moraxella catarrhalis applications, where R is H or a fatty acid and R¹ is H or α-N-acetyl-D-glucosamine or α-D-Glc.

FIG. 7. Diagram showing basal structure of carbohydrate molecule for Mannheimia haemolytica applications, where R is H or a fatty acid and R¹ is H or phosphoethanolamine.

FIG. 8. Diagram showing basal structure of carbohydrate molecule for Actinobacillus pleuropneumoniae applications, where R is H or a fatty acid and R¹ is H or phosphoethanolamine.

FIG. 9. Diagram showing basal structure of carbohydrate molecule for Pasteurella multocida applications, where R is H or a fatty acid, R¹ is H or phosphoethanolamine and R² is H or phosphoethanolamine and R³ is H or α-LD-heptose.

FIG. 10. MALDI-MS analysis of a) CRM₁₉₇ and b) galE/lpt3-CRM conjugate.

FIG. 11. SDS-PAGE and Western blot analysis of galE/lpt3-CRM conjugate; a) immediately after preparation.

FIG. 12. Whole-cell ELISA using fixed whole-cell Nm with selected rabbit sera, pre- and post-Immunisation (sera dilutions as indicated) with a) galE/lpt3 or b) lgtB/lpt3 conjugates, against fixed whole cells as indicated on the x-axes: 1=L3galE/lpt3, 2=L3icsB/lpt3, 3=L3lpt3, 4=L3icsB, 5=L3ga/E, 6=L3 wild type (wt), 7=L2gaIE, 8=L2 wt, 9=L4galE, 10=L4 wt, 11=L3lgtB/lpt3 and 12=L3lgtB. Responses as determined by OD at 405 nm at 60 mins. are shown on the y-axes. (Note: L3=strain MC58, L2=strain 35E, L4=strain 89I). The animal notation used is R for rabbit and V is used to indicate an animal that received the conjugate vaccine as opposed to a control animal.

FIG. 13. ELISA with sera from mice immunised with either icsB/lpt3-SQ-CRM (D55 #8 & #12), galE/lpt3-SQ-CRM (D56 CV5), galE/lpt3-SQ-TT (D56 TV9) or lgtB/lpt3-SQ-CRM (D49 BIV & CD3V) conjugates. Antigens are HSA, icsB/lpt3 LPS or HSA-SQ-icsB/lpt3 conjugate (a different protein, linked in the same way via squarate to carbohydrate that is not recognised (see icsB/lpt3 LPS lack of recognition)). Positive control sera icsB/lpt3-SQ-CRM (D55 #18+) confirms icsB/lpt3 LPS is on the plate.

FIG. 14. Schematic representation of the locations of fatty acid esterase (FAE) and fatty acid amidase (FAA) activities of Dictyostelium discoideum on meningococcal lipid A.

FIG. 15. Conjugation reaction scheme involving: Step 1, Dictyostelium amidase de-N-acylation; Step 2, alkaline phosphatase de-phosphorylation; Step 3, cystamine linker incorporation; Step 4, conjugation to activated protein carrier.

FIG. 16. Post-immune sera cross-reactivity ELISA against purified LPS from N. meningitidis as indicated. RVI to 4 refers to the four immunised rabbits and RC refers to the control rabbit. Dilution of each individual serum is shown in parentheses.

FIG. 17. Bactericidal assay using pre- and post-immune sera from vaccinated rabbit # 2 with Neisseria meningitidis strains MC58 galE and MC58. The percentage of serum bactericidal survival of N. meningitidis with a series of dilutions of the sera in the presence of adult serum as complement source is shown.

FIG. 18. ELISA with sera from a) mice and rabbits immunised with the cystamine linked conjugate and b) mice immunised with the direct reductive amination conjugate from a previous study [12]. Antigens are HSA, Mh losB LPS [20], meningococcal lgtB LPS or HSA-SQ-Mh IosB conjugate (a different protein, linked via squarate to a different carbohydrate). Positive control mAbs L3 B5 and Mh G3. Dilution of each individual serum is shown in parentheses.

FIG. 19. Conjugation reaction scheme for carboxyl targeting, illustrating derivitisation of Nm lgtA LPS-OH; a): Step 1, Dictyostelium amidase de-N-acylation; Step 2, protection of PEtn residue; b). Step 3, linker incorporation; Step 4, de-protection of PEtn residue; Step 5 conjugation to activated protein carrier.

FIG. 20. MALDI-MS analyses of; a) lysine targeted CRM-DTSP-galE conjugate, b) carboxyl targeted CRM-BMPH-lgtA conjugate c) carboxyl targeted CRM-ADH-SATP-lgtA conjugate.

FIG. 21. Titers of final bleed rabbit sera from immunisation with a) 25 ug of CRM-BMPH-lgtA conjugate, b) 50 ug of CRM-BMPH-lgtA conjugate and c) 28 ug of CRM-ADH-SATP-lgtA conjugate against meningococcal lgtA LPS.

FIG. 22. Cross-reactivity of final bleed rabbit sera from immunisation with a) 50 ug of CRM-BMPH-lgtA conjugate (sera diluted 1:500) and b) 28 ug of CRM-ADH-SATP-lgtA conjugate (sera diluted 1:400) against meningococcal LPS. Human serum albumin (HSA) is an irrelevant protein with or without the maleimide linker to evaluate immune response to the linker. CRM is carrier protein. Sera dilutions as indicated.

FIG. 23. Whole cell ELISA analysis of final bleed rabbit sera from immunisation with a) 50 ug of CRM-BMPH-lgtA conjugate (sera diluted 1:500) and b) 28 ug of CRM-ADH-SATP-lgtA conjugate (sera diluted 1:400) against whole cells of meningococcal strains (wt and mutants as indicated) and a control Moraxella catarrhalis (Mc lgt2) strain with sera dilutions as indicated.

FIG. 24. a) Surface labelling and b) Complement C3b deposition assays comparing pre- and post-immune sera from CRM-BMPH-lgtA immunisations with Neisseria meningitidis strains MC58 wt and lgtA mutant as antigens as indicated.

FIG. 25. Schematic diagram of two-step protein activation protocol.

FIG. 26. Titration of final bleed (D70) sera against whole cells of MhlosB, Mh wt and an irrelevant bacterial strain Nm L2 galE.

FIG. 27. Titration of pre-immune and day 70 sera against whole cells of Mannheimia haemolytica losB mutant and wt, and Moraxella catarrhalis as indicated.

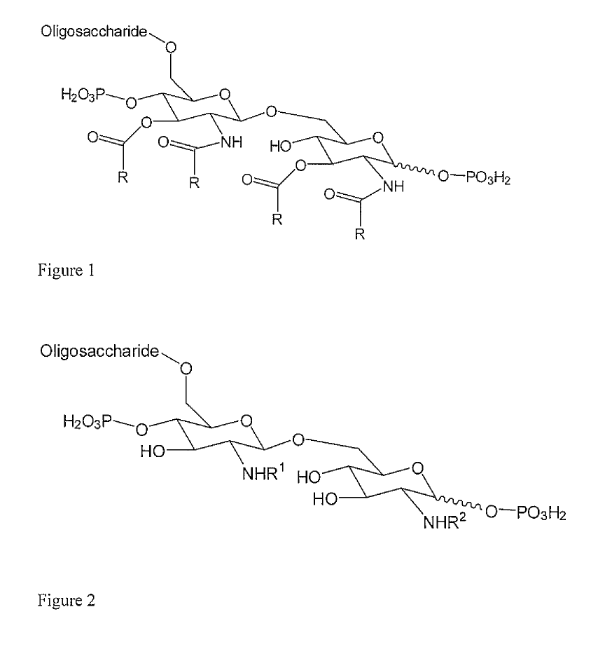

### DESCRIPTION OF THE PREFERRED EMBODIMENTS

Unless defined otherwise, all technical and scientific terms used herein have the same meaning as commonly understood by one of ordinary skill in the art to which the invention belongs. Although any methods and materials similar or equivalent to those described herein can be used in the practice or testing of the present invention, the preferred methods and materials are now described. All publications mentioned hereunder are incorporated herein by reference.

Described herein is the surprising discovery that removal of the glycosidic phosphate from the reducing end of the derived LPS molecule, to create an aldehydro functionality to be targeted for subsequent steps in the conjugation strategy, causes the formation of an immunologically dominant neo-epitope. Conjugation to the reducing end of the carbohydrate molecule following removal of the glycosidic phosphate traps the reducing glucosamine residue in an open-chain form which surprisingly was found to dominate the immune response.

Described herein are processes and antigenic structures useful in producing vaccines and compounds helpful in combating Gram-negative bacteria. Enzymes from the slime mould Dictyostelium discoideum (Dd) were used to specifically degrade O-deacylated lipopolysaccharide (LPS-OH) in such a way that a free amino functionality is created in the glucosamine disaccharide at the reducing end of the molecule, amenable for subsequent steps in the glycoconjugate production strategy, as discussed below.

Also described are strategies employed to selectively protect and de-protect important functional groups in the immunologically important region of the molecule at appropriate points in the conjugation strategy.

Also described are specific structures of the carbohydrate molecules derived using these strategies from a variety of Gram-negative bacteria, which when presented appropriately as a glycoconjugate will facilitate a functional immune response to the target core oligosaccharide region.

Numerous alternative strategies for making conjugates have been considered; ultimately a novel approach described herein was developed. This approach involves retaining the glycosidic phosphate at the reducing end of the molecule and phosphoethanolamine(s) moieties from the targeted inner core region on an LPS-derived immunogenic group having an oligosaccharide that is characteristic of a particular bacterium species. It further involves attaching this LPS-derived immunogenic group to a carrier to form a conjugate having enhanced immunogenicity. The LPS-derived group is attached to the carrier through an amine group on an amino sugar of the di-glucosamine portion of the reducing end of the molecule. The amine group is connected by a linker to a carrier, usually a protein, which is chosen to enhance immunogenicity of the immunogenic LPS-derived group. A variety of linkers can be used, and many suitable carrier proteins are known in the art. Methods for revealing the amine of the di-glucosamine, while retaining the phosphate and phosphoethanolamine groups are described herein. Methods for selectively attaching the carrier to the amine rather than to other portions of the LPS-derived portion of the conjugate are also described.

In one aspect, the invention provides an immunogenic conjugate for eliciting a specific immune response to a Gram negative bacterium having a lipopolysaccharide (LPS) endotoxin, said conjugate having the formula as shown in FIG. 2, wherein one of R¹ and R² is a linker that is attached to a carrier protein, and the other of R₁ and R² is H or a C1-C20 acyl group.

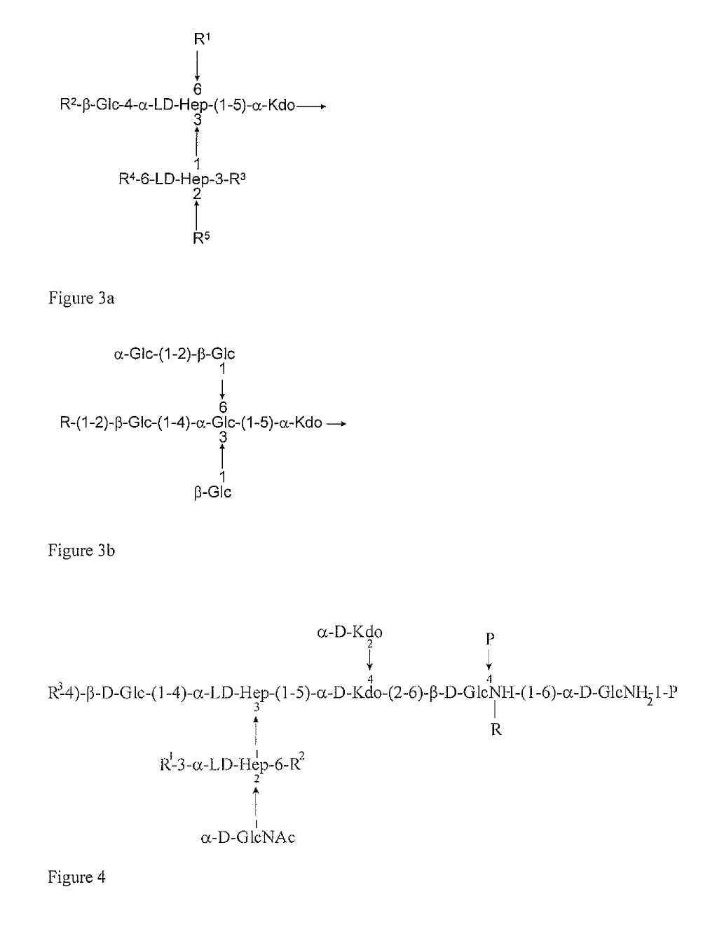

Oligosaccharide' represents at least five saccharide rings, each saccharide having a general formula as shown in FIG. 3a wherein R¹ is H or α-D-glucose; R² is H, β-D-glucose, β-D-galactose or a disaccharide of β-N-acetyl-D-glucosamine linked to the 3-position of β-D-galactose, α-DD-heptose or α-LD-heptose; R3 is H, phosphoethanolamine or α-D-glucose; R4 is H or phosphoethanolamine; and R5 is α-N-acetyl-D-glucosamine, α-LD-heptose or a disaccharide of or β-D-Glc-2-α-LD-Hep or a trisaccharide of β-D-Gal-(1-4)-β-D-Glc-2-α-LD-Hep or a tetrasaccharide of α-D-Gal-(1-4)-α-D-Gal-(1-4)-β-D-Glc-2-α-LD-Hep or a pentasaccharide of β-D-GalNAc-(1-3)-α-D-Gal-(1-4)-β-D-Gal-(1-4)-β-D-Glc-2-α-LD-Hep or in FIG. 3b where R is H or α-N-acetyl-D-glucosamine.

In one aspect, the invention provides an immunogenic conjugate for eliciting a specific immune response to a Gram negative bacterium having a lipopolysaccharide (LPS) endotoxin, said conjugate having the formula as shown in FIG. 2, wherein one of R¹ and R² is a linker that is attached to a carrier protein, and the other of R₁ and R² is H or a C1-C20 acyl group.

‘Oligosaccharide’ represents at least five saccharide rings that directly correspond to and functionally present the same conformation as the corresponding saccharide rings of the lipopolysaccharide of the bacterial endotoxin, and wherein the conjugate retains each phosphate and each phosphoethanolamine present in the corresponding portions of the natural LPS of the Gram negative bacterium.

In these immunoconjugates, the oligosaccharide contains at least 5 carbohydrate rings that correspond to the saccharide groups in the same portion of an LPS of a bacterial target. The carbohydrate rings are preferably connected to each other and to the reducing end group of Formula I in the same way they are interconnected in the natural LPS of the targeted bacterium. In many embodiments, the first of these (directly attached to the di-glucosamine portion) is Kdo. However, choosing the five or more saccharide rings in order to directly correspond to and functionally present the same conformation as the rings of a natural LPS of a targeted bacterium ensures that a biologically relevant immunological response will be produced. Optionally, more than five saccharides can be used; in some embodiments, 6 or 7 or 8 or 9, or more than 9 saccharides that directly correspond to and functionally present the same conformation as the LPS of interest are used. The oligosaccharide required may be synthesized by known methods, or it may be obtained by starting with the LPS of interest, in which case it may be obtained already connected to the reducing end di-glucosamine. Specific oligosaccharides groups are depicted and described herein for a number of bacterial targets; in each case, the oligosaccharide that is depicted can be truncated by removal of one or more saccharides in certain embodiments, provided at least five saccharide rings of the natural LPS are retained.

In each case, the oligosaccharide typically includes at least one phosphoethanolamine group (PEtn), which may be directly linked to a hydroxyl of the oligosaccharide or it may be linked to a hydroxyl through an intervening phosphate group, i.e., it may be an ethanolamine pyrophosphate in some of the LPS structures of interest. In preferred embodiments of the immunogenic conjugates of the invention, each such PEtn group is retained in the same form and location where it is found on the natural LPS.

Formula I (FIG. 2) also includes at least one carrier. The compound of formula I aside from the carrier is a relatively small molecule, and such antigens typically produce a relatively weak immunogenic response. Such antigens are typically combined with a carrier, or an adjuvant, or both to enhance their in vivo efficacy. The conjugates of Formula I are linked to a carrier that helps to increase their immunogenicity. Typically, the carrier is a protein, and frequently it is a protein known to enhance the immunogenicity of an antigen attached to it. Many such proteins are known; specific examples that can be mentioned include CRM₁₉₇; tetanus toxoid (TT), an albumin such as human serum albumin (HSA), keyhole limpet hemocyanin (KLH), polydextran, a branched polylysine core such as a MAP-4 peptide, and the like. In some embodiments, the carrier is CRM₁₉₇; in other embodiments, it is HSA, TT or KLH.

In certain embodiments of formula (I), the oligosaccharide is selected from the group consisting of Formula II, (FIG. 3a): wherein each→represents a point of attachment for the oligosaccharide to the derived lipid A region of the molecule as depicted in FIG. 2. In another embodiment of the invention, the oligosaccharide is as shown in FIG. 3a with the proviso that the oligosaccharide is not as shown in FIG. 3b.

For example, R1 may be H or α-D-glucose; R2 may be H, β-D-glucose, β-D-galactose or a disaccharide of β-N-acetyl-D-glucosamine linked to the 3-position of a β-D-galactose, α-DD-heptose or α-LD-heptose; R3 may be H, phosphoethanolamine or α-D-glucose; R4 may be H or phosphoethanolamine; and R5 may be α-N-acetyl-D-glucosamine, α-LD-heptose or a disaccharide of or β-D-Glc-2-α-LD-Hep or a trisaccharide of β-D-Gal-(1-4)-β-D-Glc-2-α-LD-Hep or a tetrasaccharide of α-D-Gal-(1-4)-β-D-Gal-(1-4)-A-D-Glc-2-α-LD-Hep or a pentasaccharide of β-D-GalNAc-(1-3)-α-D-Gal-(1-4)-β-D-Gal-(1-4)-β-D-Glc-2-α-LD-Hep.

Specific embodiments include but are by no means limited to, for example, for Neisseria meningitidis, R1 is H, R2 is H, β-D-glucose, β-D-galactose or a disaccharide of β-N-acetyl-D-glucosamine linked to the 3-position of a β-D-galactose, R3 is H, phosphoethanolamine or α-D-glucose, R4 is H or phosphoethanolamine and R5 is α-N-acetyl-D-glucosamine. For Haemophilus influenzae, R1 is H, R2 is H, R3 is H, R4 is phosphoethanolamine and R5 is α-LD-heptose or a disaccharide of or β-D-Glc-2-α-LD-Hep or a trisaccharide of β-D-Gal-(1-4)-β-D-Glc-2-α-LD-Hep or a tetrasaccharide of α-D-Gal-(1-4)-β-D-Gal-(1-4)-β-D-Glc-2-α-LD-Hep or a pentasaccharide of β-D-GalNAc-(1-3)-α-D-Gal-(1-4)-β-D-Gal-(1-4)-β-D-Glc-2-α-LD-Hep. For Mannheimia haemolytica, R1 is α-D-glucose, R2 is α-DD-heptose, R3 is H, R4 is H and R5 is α-LD-heptose. For Actinobacillus pleuropneumoniae, R1 is α-D-glucose, R2 is α-DD-heptose, R3 is H, R4 is H and R5 is α-LD-heptose. For Pasteurella multocida, R1 is α-D-glucose, R2 is H or α-LD-heptose, R3 is H or phosphoethanolamine, R4 is H and R5 is α-LD-heptose.

The compositions also include pharmaceutically acceptable salts of these compounds.

The compounds of formula (I) comprise a linker that connects the carbohydrate depicted in Formula (I) to a carrier protein. The linker can be any suitable group connecting the carbohydrate portion of formula (I) to a carrier protein. In some embodiments, the linker is a group that connects the carbohydrate and the carrier protein portions of the conjugate and is comprised of 2-40 atoms selected from the group consisting of C, S, O and N. Typically it will be a bifunctional group having as one functionality a group suitable for connection to the amine of the carbohydrate portion of formula (I), such as an acyl, formyl, or sulfonyl group. As the second functionality of the bifunctional group, the linker will typically have a group useful to connect to a moiety on the carrier protein, such as a thiol, carboxylate or amine group. Suitable second functionalities include acyl group to attach to S or N or O of the carrier protein; amine or alcohol groups to attach to a carboxylate of the carrier protein; and e.g. Michael acceptors such as an acrylate or maleimide to attach to S of the carrier protein.

In some embodiments, the linker consists of one or more groups selected from L and/or D amino acids, alkylene, alkenylene, alkynylene, heteroalkylene, heteroalkynylene, heteroalkynylene, heterocyclyl, alkyleneheterocyclylalkylene, each of which is optionally substituted with one or more substituents selected from the group consisting of alkyl, ═O, halo, COOR, CONR₂, SO₂NR₂, NRSO₂R, OR, NR₂, and CN; wherein each R is independently H or C1-C4 alkyl.

In some embodiment of formula (I), the linker connects an amine of formula (I) to a carboxylate or amine on the carrier protein, and the linker is a bond between the nitrogen of the glucosamine portion of the LPS and a carbon atom of the carrier protein, or the linker is selected from the group consisting of —(C═O)—X¹—NH—, —(C═O)—X¹—C(═O)—, a peptide linker comprising two or more amino acid moieties,

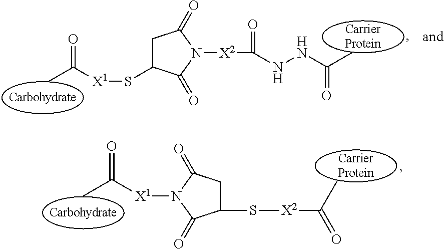

each of which may be optionally substituted, wherein X¹ and X² are each independently selected from the group consisting of C1-C8 alkylene, C1-C8 alkenylene, C1-C8 alkynylene, C1-C8 heteroalkylene, C1-C8 heteroalkenylene and C1-C8 heteroalkynylene.

In some embodiments of formula (I), R¹ is H or a C1-20 acyl group and R² is a linker that is attached to a carrier protein.

In some embodiments of formula (I), R¹ is a linker that is attached to a carrier protein and R² is H or a C1-20 acyl group.

In some embodiments of formula (I), the carrier protein is a protein that enhances the immunogenicity of the conjugate. In some embodiments of formula (I), the carrier protein is selected from the group consisting of CRM₁₉₇, tetanus toxoid (TT), human serum albumin (HSA), keyhole limpet hemocyanin (KLH), polydextran and MAP-4 peptide.

In another aspect, the invention provides a compound as shown in FIG. 2, wherein said carrier protein is selected from the group consisting of CRM₁₉₇, tetanus toxoid (TT), human serum albumin (HSA), keyhole limpet hemocyanin (KLH), polydextran and MAP-4 peptide;

Oligosaccharide represents one or more oligosaccharides containing at least five contiguous saccharide rings of a moiety selected from the group consisting of Formula II (FIG. 3) set forth above, wherein one of R¹ and R² is a linker that is attached to a carrier protein, and the other of R₁ and R² is H or a C1-C20 acyl group and said linker is selected from the group consisting of a bond between the nitrogen of the glucosamine portion of the LPS and a carbon atom of the carrier protein, (C═O)C1-C8 alkylene, (C═O)C1-C8 alkenylene, (C═O)C1-C8 alkynylene, C1-C8 alkylene(C═P), C1-C8 alkenylene(C═O), C1-C8 alkynylene(C═O), (C═O)C1-C8 heteroalkylene, (C═O)C1-C8 heteroalkenylene, (C═O)C1-C8 heteroalkynylene, a peptide linker, C2-C20 poly(ethylene glycol),

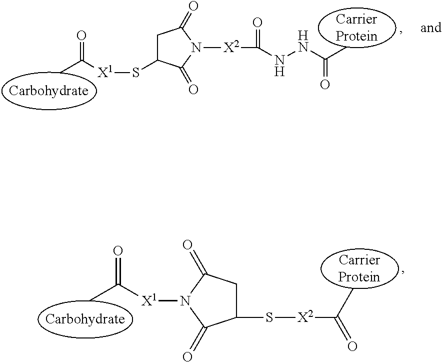

wherein the linker is the group connecting the carbohydrate and the carrier protein portions of the conjugate; and

X¹ and X² are each independently selected from the group consisting of C1-C8 alkylene, C1-C8 alkenylene, C1-C8 alkynylene, C1-C8 heteroalkylene, C1-C8 heteroalkenylene and C1-C8 heteroalkynylene.

In some embodiments, Oligo is selected from the group consisting of Formula II as set forth above.

In another aspect, the invention provides an immunogenic conjugate for eliciting a specific immune response to a Gram negative bacterium having a lipopolysaccharide (LPS) endotoxin, said conjugate having at least 5 molecules of the carbohydrate depicted as

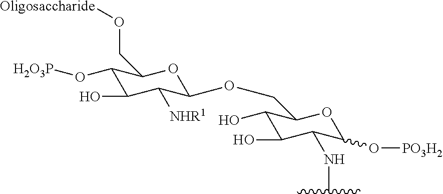

is conjugated to a single carrier protein via a linker;
wherein the oligosaccharide, carrier protein, linker and R¹ are as defined above for formula (I). It is understood that in formula (I), the carrier protein may have more than one LPS-derived group attached to it, i.e., a single carrier protein can have 5 or more, or 8 or more, and preferably 10 or more of these LPS-derived groups attached. In some embodiments, there can be 5-10 LPS-derived groups attached to one carrier; or 10-15 such groups attached to one carrier; or 10-20, or 20-30, or 30-40 LPS-derived groups linked to a single carrier protein molecule. In some embodiments, the conjugate for eliciting a specific immune response to a Gram negative bacterium having a lipopolysaccharide (LPS) endotoxin is depicted as

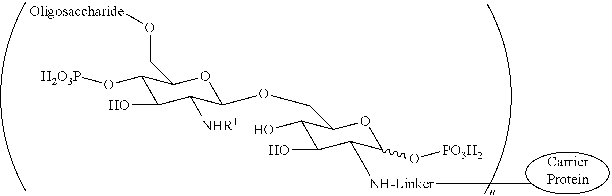

wherein n is at least 5;
and oligosaccharide, carrier protein, and R¹ are as defined for formula (I).

As will be appreciated by one of skill in the art, in this manner, more than one oligosaccharide molecule will be attached per carrier protein.

In another aspect, the invention provides an immunological composition comprising a conjugate of formula (I) admixed with at least one vaccine adjuvant.

In another aspect, the invention provides a method to elicit a specific immune response to a Gram negative bacterium, comprising administering to a subject in need of such treatment an effective amount of the conjugate of formula (I) or a vaccine composition comprising at least one such conjugate.

In another aspect, the invention provides a method to make an LPS-based immunological conjugate that induces an immune response effective against a Gram negative bacterium, comprising the steps of:

obtaining a lipopolysaccharide (LPS) from the Gram negative bacterium; removing acyl groups linked to oxygen on the di-glucosamine of the Lipid A portion of the LPS;

removing at least one acyl group linked to N of the di-glucosamine reducing end of the LPS to provide an amine group;

protecting at least one phosphoethanolamine group attached to the oligosaccharide portion of the LPS:

attaching one end of a linking group to an amine group on the di-glucosamine;

attaching the other end of the linking group to a carrier moiety; and

de-protecting at least one phosphoethanolamine.

In some embodiments, wherein the LPS-derived moiety comprises a phosphate on the anomeric center of the reducing glucosamine moiety of the LPS, and the phosphate is retained in the immunogenic conjugate. Typically, a second phosphate group is present on the reducing di-glucosamine, and this second phosphate is also retained in the conjugate. In some embodiments, each phosphate and each phosphoethanolamine of the LPS from the Gram negative bacterium is preserved in the immunogenic conjugate.

The step of removing at least one acyl group from N of the di-glucosamine portion of the LPS can be done by any suitable method that does not remove the anomeric phosphate of the reducing end glucosamine. In some embodiments it is done enzymatically. In some embodiments, this is done contacting the LPS or modified LPS with an amidase that selectively removes the acyl group. In one embodiment, the amidase is an amidase from Dictyostelium discoideum.

In the method described, when the LPS-derived moiety includes at least one phosphoethanolamine group (which can be a pyrophosphoethanolamine), the method is adapted to preserve this PEtn group in the immunogenic conjugate because that increases the immunogenicity of the conjugate. The PEtn group must be distinguished from the amine(s) of the reducing end of the LPS-derived portion of the conjugate, though, to avoid attachment of the linker to the phosphoethanolamine. This can be accomplished by any suitable method, but in some embodiments it is achieved by use of selective protecting group chemistry. Such chemistry is well known, see e.g. TH Greene's book, PROTECTIVE GROUPS IN ORGANIC SYNTHESIS (1980). Methods for removing the protecting group at an appropriate time are also well known in the art.

In some embodiments, the phosphoethanolamine group is selectively protected using a carbamate group. The selective protection step may be performed before or after treatment with an amidase to remove one or more of the fatty acids from the amine group(s) of the reducing di-glucosamine portion of the derived LPS molecule. It has been demonstrated that it can be done selectively after deacylation with an amidase using certain carbamate protecting groups; in some embodiments the amine is protected as a methyl carbamate, t-butyl carbamate or benzyl carbamate.

The methods described herein can be applied to any bacterial lipopolysaccharide of a Gram negative bacterium that comprises at least five saccharide rings attached to a di-glucosamine portion at the reducing end of the derived LPS molecule, so that a compound of formula (I) can be prepared. In some embodiments, the Gram negative bacterium is Neisseria meningitidis. In some embodiments, the Gram negative bacterium is Haemophilus influenzae. In some embodiments, the Gram negative bacterium is Moraxella catarrhalis. In some embodiments, the Gram negative bacterium is Mannheimia haemolytica. In some embodiments, the Gram negative bacterium is Actinobacillus pleuropneumoniae. In some embodiments, the Gram negative bacterium is Pasteurella multocida.

In yet another aspect, the invention provides an LPS-based immunological conjugate prepared by the method described above, and preferably one where the Gram negative bacterium is selected from Neisseria meningitidis, Haemophilus influenzae, Moraxella catarrhalis, Mannheimia haemolytica, Actinobacillus pleuropneumoniae, and Pasteurella multocida. In some embodiments, this conjugate is admixed with at least one vaccine adjuvant, to enhance its effectiveness. Typically, it is formulated as a vaccine that can further comprise additional adjuvants, other immunogens, and various stabilizing and preservative compositions known in the art.

In yet another aspect, the invention provides a method to elicit a specific immune response to a Gram negative bacterium, comprising administering to a subject an effective amount of the conjugate described above, or a vaccine composition comprising the conjugate described above.

The vaccine adjuvant for use in these compositions can be any substance that further enhances the immunogenic response elicited by an immunoconjugate of the invention. Suitable adjuvants known for such effects include. e.g., Freund's complete or incomplete adjuvant and other oil-in-water emulsions; liposomes; saponin and ISCOMs based on it, which may include, e.g., components from influenza, measles, rabies, gp340 from EB-virus, gp120 from HIV, Plasmodium falciparum and Trypanosoma cruzi; DETOX (Ribi Immunochemicals); Montanide ISA-51, -50, and -70; QS-21, monophosphoryl Lipid A; squalene compositions; alum and aluminum phosphate; bacterial products such as Bordetella pertussis components, Corenybacterium-derived P40 component, cholera toxin and mycobacteria; and the like. The composition can further contain one or more additional pharmaceutical excipients, such as suitable medium (e.g., sterilized isotonic aqueous solution), buffers, stabilizers, and preservatives such as thimerosal.

Where the invention comprises a method to elicit a specific immune response to a Gram negative bacterium, this can comprise administering to a subject an effective amount of the conjugates of the invention or a vaccine composition comprising the conjugates as described above. Methods to determine an effective amount are known in the art, and can comprise assessment of the amount required to induce formation of relevant antibodies in a subject, as well as systematic studies to determine an amount suitable for an average person, to reduce susceptibility to infection by the bacterium of interest by at least 50% or by at least 75%, or by at least about 90%. It is well known that vaccine compositions are not necessarily 100% effective in all subjects; however, it is also widely accepted that a statistically significant reduction in likelihood of contracting an infection has tremendous public health benefits to slow the spread of contagious infections. Thus an effective amount, as used herein, is an amount that reduces susceptibility to an infecting bacterium by at least about 50%, and preferably by at least 75%.

Suitable subjects for the present immunoconjugates include mammalian and avian species, including horses, cows, goats, pigs, and other farm animals; chickens, turkeys, ducks and geese; and dogs and cats and other domestic pets. Humans are a preferred subject.

Administration of the immunoconjugates of the invention can be done using any suitable method. Oral or parenteral delivery are contemplated, as well as topical, buccal, nasal, or suppository. Parenteral administration is sometimes preferred, and may be systemic or local. Suitable formulations for each route of administration are known in the art.

An immunoconjugate of the invention may be administered once, or more than once. In some embodiments, an initial dosage is administered, and a later booster dosage is also administered. Where a booster is required, it may be administered about a week after the initial dosage; or about a month to two months after the initial dosage, or about 3-6 months after the initial dosage, or 1-2 years after the initial dosage.

Where the invention provides a method to make an LPS-based immunological conjugate that induces an immune response effective against a Gram negative bacterium, the method comprises at least some of the following steps:

1. obtaining a lipopolysaccharide (LPS) from the Gram negative bacterium of interest;

2. removing acyl groups linked to oxygen on the di-glucosamine of the Lipid A portion of the LPS ; typically, this step is done chemically, using e.g. hydrazine or other suitable alkaline solution.

3. removing at least one acyl group linked to N of the di-glucosamine of the reducing end of the LPS to provide an amine group; this step can be done chemically or enzymatically.

4. protecting where necessary at least one phosphoethanolamine group attached to the oligosaccharide portion of the LPS: this is often done by acylating the PEtn group to convert its amine into a carbamate, such as a BOC or CBZ carbamate.

5. attaching one end of a linking group to the amine group on the di-glucosamine; (this involves attaching a linker to an amine liberated by the deacylation of N in step 3 above. Suitable reactions for attaching the linker include acylation, alkylation, sulfonylation, and reductive amination.

6. de-protecting where necessary at least one phosphoethanolamine and attaching the other end of the linking group to a carrier moiety.

The linking step involves reacting a functional group on the linker with a suitable functionality on the carrier moiety (protein, typically). The functionality on the carrier moiety may be a naturally occurring carboxylate, amine, or thiol of a protein carrier, for example; or it may be one of these or a similar reactive functionality that has been introduced onto the carrier for the purpose of reacting with the linker. In one embodiment, for example, the linker and carrier are joined together by a Michael reaction between a thiol and a strong Michael acceptor such as a maleimide. The maleimide can be attached to either the LPS-derived portion of the conjugate, or to the carrier portion of the conjugate; and the thiol is attached to the other one. In preferred embodiments, the thiol is attached to the carrier protein, or is a natural component of the carrier protein, and a maleimide or similar Michael acceptor is attached to the amine of the Lipid A moiety. In other embodiment, the maleimide or other Michael acceptor such as an acrylate, is attached to the LPS-derived portion of the conjugate, and the thiol is on the carrier portion of the conjugate.

In some embodiments, all of the foregoing steps are used.

Suitable oligosaccharide groups for each of these species are disclosed in the present application. As an example, an immunoconjugate for N. meningitidis could have the following structure:

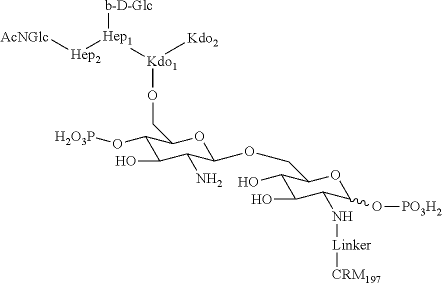

As should be apparent, Kdo1 and Kdo2 and Hep1 and Hep2, and beta-D-Glc and AcNGlc each represent a saccharide group that directly corresponds to and functionally presents the same conformation as the corresponding groups from the natural LPS portion disclosed herein, including their phosphorylation or phosphoethanolamine substitution patterns. Preferably, each of these groups is the same as the corresponding group in the natural oligosaccharide portion of the N. meningitidis LPS, and all of them are linked together in the same fashion as the corresponding rings of the natural LPS.

In one aspect, the invention provides an immunogenic conjugate comprising an LPS-derived group linked to a carrier, and methods to make such immunogenic conjugates that contain a lipopolysaccharide group of a Gram-negative bacterium. The immunogenic conjugate comprises an oligosaccharide portion that is derived from a Gram negative bacterium and thus confers an immune response targeting the particular bacterium whose oligosaccharide is used. It also contains a carrier protein that is included to enhance immunogenicity of the oligosaccharide portion, and a linker that connects the carrier protein to the oligosaccharide.

The methods of the invention comprise removing O-linked fatty acids from the di-glucosamine portion of a Lipid A moiety of the LPS of a Gram negative bacterium, and removing at least one acyl group from an amine of the di-glucosamine group to produce a modified LPS. An amine on the di-glucosamine at the reducing end of the modified LPS is then used as a connection point for attaching a suitable carrier moiety to the modified LPS.

Many Gram negative bacterial LPS structures contain a phosphoethanolamine (PEtn) group on their oligosaccharide portion. It has been observed that immunogenic conjugates based on bacterial LPS structures may produce weaker immunological responses to the native structure if the original phosphoethanolamine group(s) is lost when the conjugate is prepared. The methods of the invention therefore also provide a protection strategy that permits phosphoethanolamine groups on the oligosaccharide to be retained. Furthermore, the methods utilize conditions that permit the glycosidic phosphate group typically present at the reducing end of the molecule to be retained. The method thus leaves phosphoethanolamine and phosphate groups of the natural LPS intact, while attaching a carrier to the modified LPS through an amine of the di-glucosamine portion of the reducing end of the molecule.

The methods thus provide an immunogenic conjugate that retains a species-specific oligosaccharide portion of an LPS, including phosphate and phosphoethanolamine groups. Because it retains these features and minimizes alteration of the reducing end of the molecule, it provides an effective immunogenic conjugate that produces immunogenic response in a subject treated with the conjugate or a vaccine composition containing it. Moreover, because the described methodology facilitates the retention of any native phosphoethanolamine groups, it produces a strong immunogenic response directed to conserved inner core regions that may elaborate phosphoethanolamine moieties on the native LPS of the targeted Gram negative bacterium. And because of the method used to link the modified LPS to the carrier, the linking process and structure do not induce a separate significant immunogenic response that interferes with the effectiveness of the conserved region: other methods of linking an oligosaccharide portion of an LPS to a carrier through a more substantially altered reducing end of the LPS molecule, have been found to induce non-productive immune responses directed to the linking groups or modifications of the LPS molecule (neo-epitopes introduced by the linking process or to the structure). Thus the conjugates of the invention provide a far more effective immunogenic conjugate than ones produced by conjugation methods that attach the carrier through a carboxylate of a Kdo group of the oligosaccharide, or methods that cause loss of the natural phosphate and/or phosphoethanolamines on the LPS, or methods (as described herein) that open one of the glucosamine rings of the di-glucosamine portion of the reducing end of the LPS molecule and thus produce an interfering neo-epitope that dominates the immune response to the conjugate.

In another aspect, the invention provides an immunogenic conjugate made by the method described above, or an immunogenic conjugate made to meet the structural requirements above.

In another aspect, the invention provides an immunogenic composition comprising an immunogenic conjugate as described herein and at least one vaccine adjuvant to further enhance immunogenicity. Such adjuvants are known in the art.

The linker can be any compatible linker that is suitable for attachment to an amine group. The linker comprises two functional groups: one of these groups is adapted to react with the amine of the amino sugar, and the other is adapted to react with an available functional group on the carrier. The functional group on the carrier can be an amine, carboxylate, thiol or alcohol group, or it can be a functional group that is added to the carrier by a connector moiety. The connector moiety can be any small molecule having one functional group selected for its ability to form a covalent bond to the carrier, and a second functional group selected to be compatible with and form a bond to the second functional group of the linker.

According to an aspect of the invention, there is provided a method of preparing a glycoconjugate comprising: separating lipopolysaccharide from a bacterium of interest; de-esterifying the lipopolysaccharide; removing at least one N-linked fatty acid from the de-esterified carbohydrate molecule with an isolated amidase activity; protecting phosphoethanolamine residue(s) on the carbohydrate molecule with a selective blocking group; incorporating a linker molecule at the amino functionality of the reducing glucosamine residue of the carbohydrate molecule; de-protecting the phosphoethanolamine residue(s) of the carbohydrate molecule; and conjugating the derived carbohydrate molecule to a suitably activated carrier molecule. The glycoconjugate may be used for generating an immune reaction, for example, as a vaccine.

The bacterium may be a Gram-negative bacterium. Specific embodiments of the invention relate to Neisseria meningitidis, Haemophilus influenzae, Moraxella catarrhalis, Mannheimia haemolytica, Actinobacillus pleuropneumoniae, and Pasteurella multocida. Each of these bacteria has a cell wall that comprises an LPS group that is characteristic of the bacterium and is useful as an immunogen for vaccine development. However, producing an effective, broad spectrum immunogen for such bacteria is complicated by several factors. First, the LPS groups on these bacteria include a toxic Lipid A portion; to produce a safe and effective vaccine, this toxicity must be mitigated. Second, the LPS includes many different functional groups, including phosphates, phosphoethanolamines, carboxylates, fatty acids, etc. in addition to a number of carbohydrate rings linked together; in order to make an effective vaccine, the immunogenic group must be chosen to include the correct ones to provide specific recognition and high affinity. Additionally, in order to elicit a strong immune response, the LPS-derived immunogen must be linked to a carrier, typically a protein, to enhance its effect; that requires selecting an attachment point for the carrier from among the many possible options on the highly functionalized LPS moiety. The present invention addresses each of these issues, and provides a general method for making useful immunogenic conjugates from LPS groups of Gram negative bacteria. Glycoconjugates of the invention comprise a lipopolysaccharide-derived component linked by a linker to a carrier. The carrier may be a protein. Proteins useful as carriers for bacterial immunogenic compositions are well known in the art; specific examples useful with the present conjugates include but are not limited to CRM₁₉₇, tetanus toxoid (TT) and HSA.

The carbohydrate molecule may be conjugated to an activated carboxyl or amino group on the protein carrier. Alternatively, it can be conjugated to a free thiol or hydroxyl group on the carrier. Where the carrier has multiple available functional groups, it is possible to attach two or more LPS-derived moieties to one carrier rather than just a single one. In some embodiments, 5 or more LPS-derived moieties are attached to a single molecule of the carrier, e.g., CRM₁₉₇. In other embodiments, at least ten LPS-derived moieties are attached to a single molecule of a carrier protein.

The selective blocking group may be any conventional amine protecting group that is compatible with the conditions used to link the carbohydrate moiety to the carrier, and is also readily cleaved from the amine under conditions that do not damage the conjugate. One example of a suitable protecting group is t-butoxycarbonyl, also called a Boc group. Other amine protecting groups that are removable under similarly mild conditions can also be used, for example carbobenzyloxy (Cbz) or other benzyl carbamates can be used; these can be removed under convenient and mild hydrogenolysis conditions. Similarly, methoxycarbonyl groups can be used, and can be deprotected under mild conditions, e.g. using BBr₃, Me₂BBr, or TMSI.

The linker connecting the carbohydrate molecule to the carrier may be Sulfo-GMBS. The linkage between the groups is typically formed by attaching a bifunctional group to either the LPS-derived portion or to the carrier portion of the conjugate; or one bifunctional group can be attached to the LPS-derived carbohydrate moiety and another bifunctional group can be attached to the carrier, and the two bifunctional groups can then react to bond to each other.

Any suitable bifunctional group can serve as the linker between the LPS-derived group and the carrier. The carrier molecule may be activated by incorporation and reduction of PDPH. The carrier molecule may be activated by incorporation of adipic dihydrazide and SATP.

While certain structures are depicted for convenience herein as neutral species, e.g. phosphate groups are sometimes depicted as —OPO₃H₂, the invention includes the salts of these groups, which may be formed by conventional methods for use, and which may be produced in vivo according to the pH of the environment in which the compounds are utilized.

According to another aspect of the invention, there is provided a conjugate comprising a carrier molecule covalently linked to a carbohydrate molecule that comprises a conserved structure and is capable of provoking a cross-reactive immune response against heterologous strains of the targeted bacterial species. The carbohydrate molecule is generally derived from a conserved inner core lipopolysaccharide of the target species, said inner core being conserved and thus capable of presenting epitopes elaborated by wild-type strains of the target species.

The carrier molecule may be a protein.

The carbohydrate molecule may be covalently linked to the activated carboxyl groups or amino groups of the protein carrier.

The protein may be for example but is by no means limited to CRM₁₉₇ or TT.

The oligosaccharide or carbohydrate portion of the conjugates of the invention is selected to correspond to the carbohydrate portion of a bacterial LPS. A particular carbohydrate group can be used to produce an immune response against a particular bacterium by copying part or all of the carbohydrate portion of the LPS produced by that bacterium. Typically, at least 5 and preferably six or more of the inner core and/or outer core saccharide rings of the bacterial LPS are included with the Lipid A moiety when constructing the modified LPS portion of the conjugates described herein. For applications to combat diseases caused by Neisseria meningitidis the carbohydrate molecule may comprise a D-glucose, two LD-heptose, a N-acetyl-D-glucosamine, two Kdo, two D-glucosamine and two phosphate residues, comprising the basal structure shown in FIG. 4, where R is H or a fatty acid, R¹ is H, phosphoethanolamine or β-D-glucose, R² is H or phosphoethanolamine and R³ is H, β-D-glucose, β-D-galactose or a disaccharide of β-N-acetyl-D-glucosamine linked to the 3-position of a β-D-galactose.

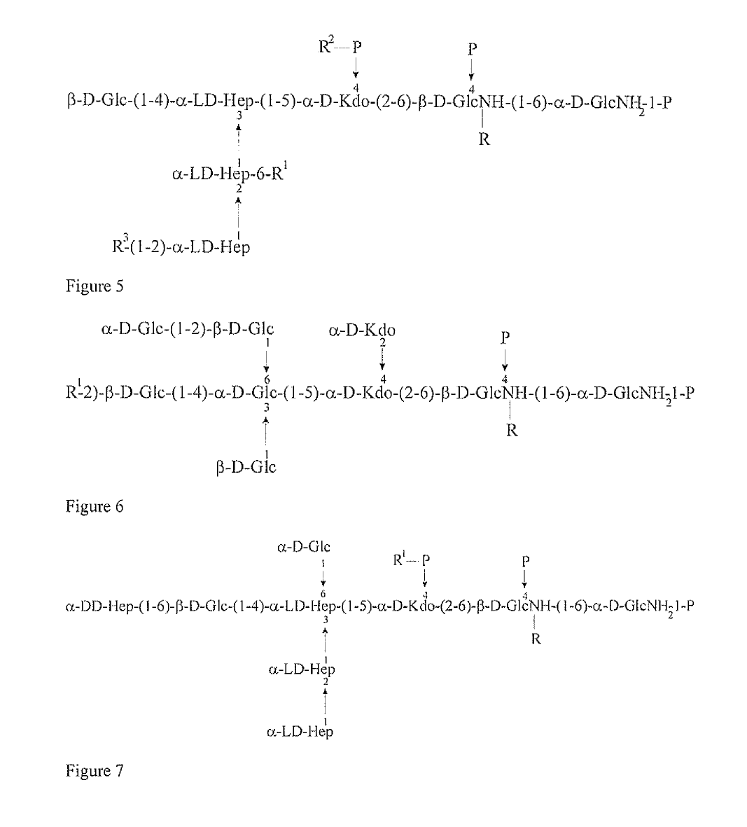

For applications to combat diseases caused by Haemophilus influenzae the carbohydrate molecule may comprise a O-glucose, three LD-heptose, one Kdo, two O-glucosamine and three phosphate residues, comprising the basal structure shown in FIG. 5, where R is H or a fatty acid, R¹ is H or phosphoethanolamine, R² is H or phosphoethanolamine and R³ is H or β-D-Glc or a disaccharide of β-D-Gal-(1-4)-β-D-Glc or a trisaccharide of α-D-Gal-(1-4)-β-D-Gal-(1-4)-β-D-Glc or a tetrasaccharide of)-β-D-GalNAc-(1-3)-α-D-Gal-(1-4)-β-D-Gal-(1-4)-β-D-Glc.

For applications to combat diseases caused by Moraxella catarrhalis the carbohydrate molecule may comprise a five D-glucose, two Kdo, two D-glucosamine and two phosphate residues, comprising the basal structure shown in FIG. 6, where R is H or a fatty acid and R¹ is H or α-N-acetyl-D-glucosamine or α-D-Glc.

For applications to combat diseases caused by Mannheimia haemolytica the carbohydrate molecule may comprise two D-glucose, three LD-heptose, a DD-heptose, a Kdo, two D-glucosamine and three phosphate residues, comprising the basal structure as shown in FIG. 7, where R is H or a fatty acid and R¹ is H or phosphoethanolamine.

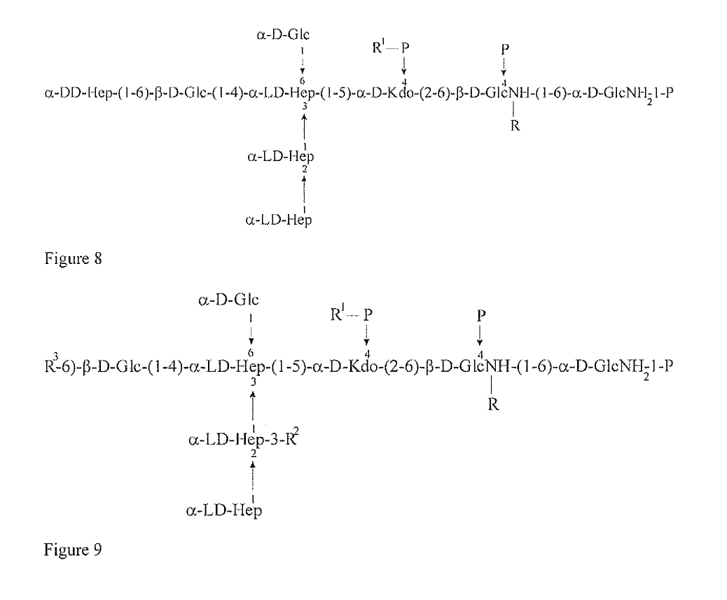

For applications to combat diseases caused by Actinobacillus pleuropneumoniae the carbohydrate molecule may comprise two O-glucose, three LD-heptose, a DD-heptose, a Kdo, two D-glucosamine and three phosphate residues, comprising the basal structure shown in FIG. 8, where R is H or a fatty acid and R¹ is H or phosphoethanolamine.

For applications to combat diseases caused by Pasteurella multocida the carbohydrate molecule may comprise two O-glucose, three LD-heptose, a Kdo, two O-glucosamine and three phosphate residues, comprising the basal structure shown in FIG. 9, where R is H or a fatty acid, R¹ is H or phosphoethanolamine and R² is H or phosphoethanolamine and R³ is H or α-LD-heptose.

For each of these bacterial species more of the saccharides of the natural LPS can also be included; however, for purposes of the invention, the portions shown attached to the reducing end of each LPS molecule are believed to be adequate to elicit a strong and specific immunogenic response.

Described herein is a novel strategy which avoids the creation of an unwanted immunodominant open chain structure, but maintains a strategy where the protein carrier is still attached via the glucosamine disaccharide. Also described is a novel blocking and unblocking strategy which protects crucial residues in the core oligosaccharide region during the conjugation methodologies. Also described are antigenic carbohydrate structures derived from LPS by utilising the detailed methodologies, which when appropriately presented on the carrier molecule will provoke a functional immune response. The structures and methodologies will be illustrated in the following series of examples. The examples are intended to be illustrative and do not limit the invention.

### Example 1

### Identification of Open Chain Neo-Epitope

In previous studies using Neisseria meningitidis serogroup B (NmB) glycoconjugates prepared from O-deacylated lipopolysaccharide (LPS-OH), we showed that this vaccine could elicit protective antibodies against invasive NmB disease. These LPS-OH glycoconjugates proved difficult to prepare because the presence of amide linked fatty-acyl groups results in glycolipids that are relatively insoluble and aggregate. Therefore, we studied the immunogenicity of Nm glycoconjugates utilising completely deacylated LPS. LPS from mutants of Nm (MC58 galE/lpt3 or icsB/lpt3 or lgtB/lpt3) was covalently linked to a carrier protein (CRM₁₉₇, TT, HSA) using a stepwise process involving complete deacylation, enzymic de-phosphorylation, amination and coupling to the carrier protein using squarate chemistry.

The resulting conjugates were characterised by SDS-PAGE and Western blots with a carbohydrate-specific monoclonal antibody and by colorimetric assays. Following immunisation, sera from mice (BALB/c and CD1) and New Zealand white rabbits were assayed by ELISA for their reactivity to LPS or to whole cells of homologous and heterologous Nm strains. The immune responses in mice were inconsistent. In contrast, each of 5 rabbits vaccinated with galE/lpt3-CRM or lgtB/lpt3-CRM resulted in high IgG titres. The inconsistent immunogenicity of these conjugates was found to be due to the creation of a “neo-epitope” during the conjugation procedure. The “neo-epitope” was determined to be the open chain glucosamine residue created by removal of the glycosidic phosphate, which was found to be immunodominant, diminishing or completely precluding an immune response to the target region of the conjugated carbohydrate. The minority of antibodies to the target oligosaccharide region only recognised LPS of the homologous strain or heterologous strains that lacked a phosphoethanolamine attached to the 3-position of the distal heptose residue (Hep II) of the inner core (designated PEtn-3), or that lacked significant oligosaccharide extension from the proximal heptose residue (Hep I). In contrast, wild-type strains that elaborated PEtn-3 or had oligosaccharide extension from Hep I were not recognised by these sera. Similar patterns of reactivity were found in assays in which binding of antibody to live whole-cells adherent to monolayers of epithelial cells was determined using immunofluorescence microscopy.

These studies demonstrated that whereas the methodology to prepare conjugates from O- and N-deacylated LPS was robust the conjugates failed to elicit the type of high affinity antibodies required to protect against the majority of wild-type NmB disease isolates. We conclude that to attain optimal immunogenicity, conjugates should retain PEtn in the LPS molecule following deacylation and avoid the immunodominance of the open-chain neo-epitope.

### Production and Characterisation of Conjugates

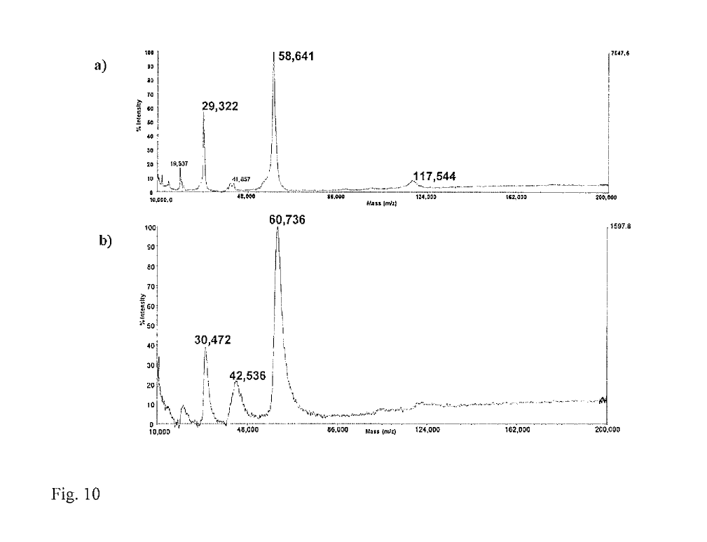

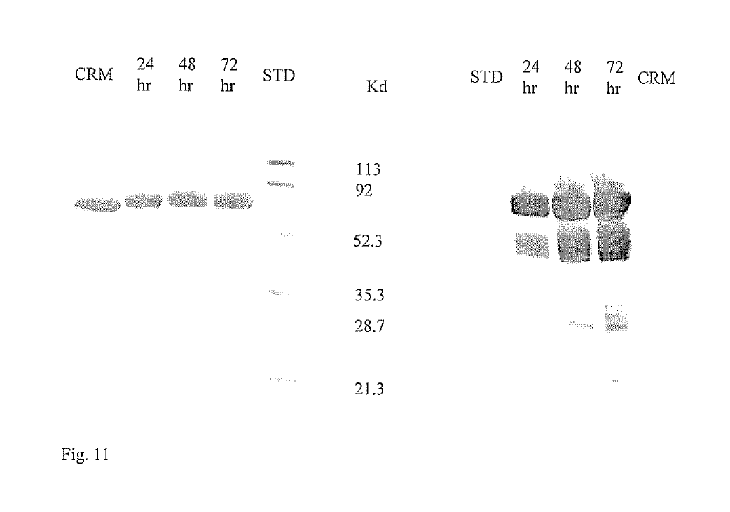

Conjugates were prepared according to a strategy that included complete de-acylation of the lipid A region, removal of the glycosidic phosphate, amination and conjugation to the protein moiety via squarate linker chemistry. The purified conjugates were examined by MALDI-MS (FIG. 10) and SDS-PAGE with Western blotting with a carbohydrate specific monoclonal antibody LPT3-1 (FIG. 11a) [15]. MALDI-MS analysis of non-conjugated CRM₁₉₇ gave a sharp singly charged ion at 58,641 Da consistent with the published molecular weight for this protein (FIG. 10a). MALDI-MS analysis of the galE/lpt3-CRM₁₉₇ conjugate gave a broader peak with a maximum at 60,736Da (FIG. 10b). Although, this mass is not consistent with the presence of a whole number of carbohydrate molecules, a similar mass gain was consistently observed for each conjugate produced, indicating additions of at least one carbohydrate moiety.

### Immunogenicity of Conjugates

### LPS ELISA

Mice (all conjugates) and rabbits (galE/lpt3 and lgtB/lpt3 CRM₁₉₇ conjugates) were immunised. None of the control immunisations in rabbits or mice resulted in an IgG response to the carbohydrate moiety. The number of mice responding to the CRM₁₉₇ conjugates varied from 66% for icsB/lpt3 to 30% for the galE/lpt3 and lgtB/lpt3 conjugates whereas all rabbits responded to the galE/lpt3 and lgtB/lpt3 CRM₁₉₇ conjugates. Nevertheless, those animals that did respond showed a strong immune response to the immunising carbohydrate antigen when the sera were examined for cross-reactivity with homologous and heterologous LPS by ELISA. Essentially, a strong response was observed with the immunising antigen, but the presence of phosphoethanolamine at the 3-position of the distal heptose residue (Hep II) and I or the degree of extension from the proximal heptose residue (Hep I) tended to preclude recognition with the truncated, phosphoethanolamine-lacking conjugate derived sera.

### Whole Cell ELISA

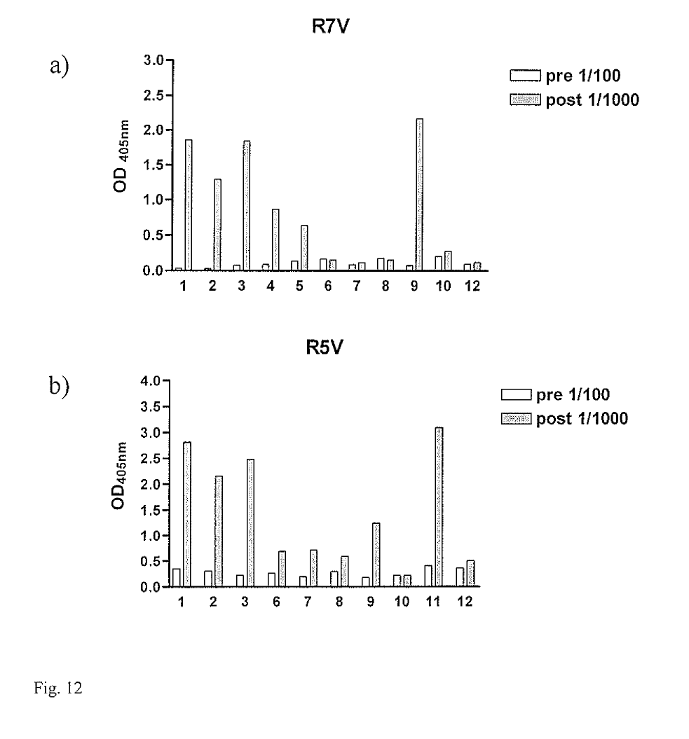

Using sera from galE/lpt3 conjugates in rabbits (FIG. 12a) and lgtB/lpt3 conjugates in rabbits (FIG. 12b) pre-vaccination sera and post-immune sera were compared for their ability to bind to whole-cells by ELISA. This demonstrated that sera from mice and rabbits immunised with the conjugates were able to bind to whole-cells of the conjugate mutant but not to wild-type strains that elaborate significant “alpha-chain” extension from Hep I or elaborate a PEtn residue at Hep II.

### Identification of a Neo-Epitope Created During the Conjugation Methodology

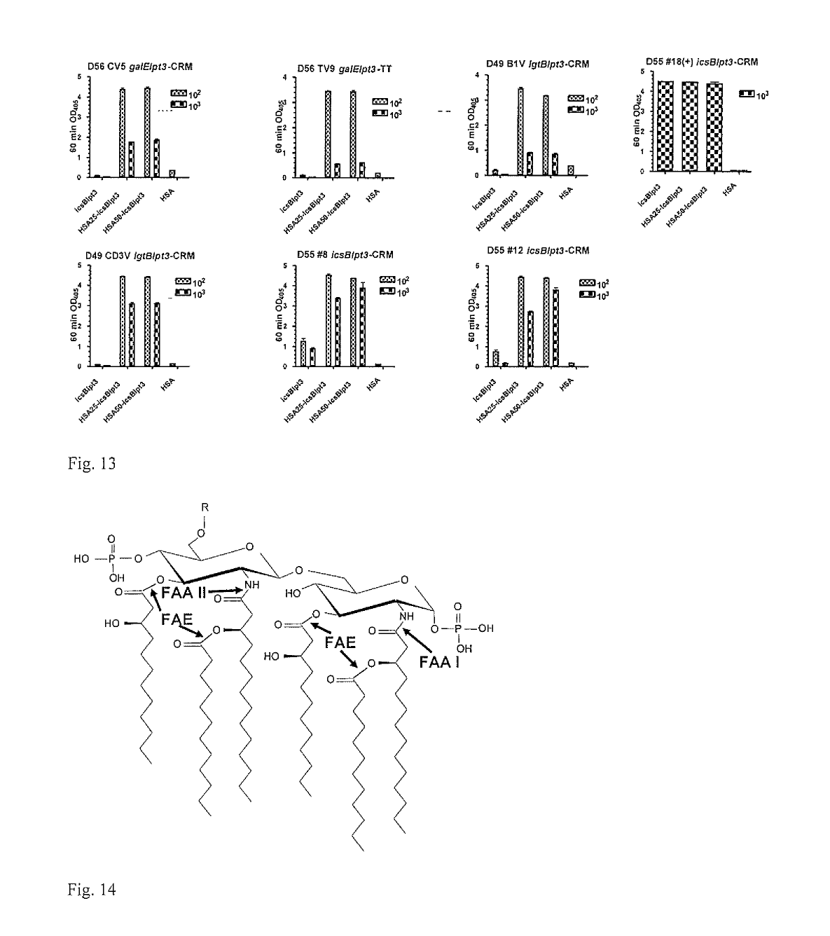

To examine if conjugation of the carbohydrate molecule is inadvertently creating immunodominant “neo-epitopes”, experiments were conducted to examine whether sera from conjugate immunisations were recognising the linker molecule or other regions of the conjugated carbohydrate in unrelated antigen substrates. Studies were performed with sera from galE/lpt3-(or icsB/lpt3 or lgtB/lpt3)-SQ-CRM conjugates against icsB/lpt3-SQ-HSA. Squarate itself is not being recognised, as sera from the conjugates prepared in this study did not recognise irrelevant antigens containing the squarate linker as the only common entity, suggesting that the linker itself is not dominating the immune response. However, subsequent studies clearly indicated that a neo-epitope was created on the carbohydrate by virtue of the conjugation. This neo-epitope consists of at least the open-chain sugar created following alkaline phosphatase treatment, and then “trapped” in the open chain form following reductive amination with ammonium acetate prior to squarate linker addition. Conjugates with unrelated carbohydrate, linkers and proteins to the sera being tested were used as antigens, and recognition was observed due to the only common feature, namely the open chain glucosamine residue (FIG. 13).

### Example 2

### Open-Chain Neo-Epitope is Immunodominant

A further study developed the use of amidases produced by the slime mould Dictyostelium discoideum, which remove N-linked fatty acids from the lipid A region of the LPS molecule without further modification to the core oligosaccharide, and most importantly enable the retention of the PEtn residue. We examined two approaches utilizing the Dictyostelium amidases. Firstly, we attempted to “hide” the neo-epitope discussed above by developing a conjugation strategy wherein the carrier protein was conjugated directly to the carbohydrate molecule without the use of a linker, akin to the approach that had shown success in earlier studies with O-deacylated LPS. This conjugation strategy however was very inefficient and did not attach sufficient carbohydrate to the protein carrier using this methodology to provoke an immune response to the carbohydrate. Secondly, we theorized that with the retention of the immunologically important PEtn residue the immune response to the neo-epitope would not be so deleterious. Using a cystamine linker strategy we obtained broadly cross-reactive antisera following rabbit immunizations which were capable of recognising fully extended wild-type strains that elaborated a PEtn residue at the 3-position of the distal heptose residue of the inner core oligosaccharide. Although bactericidal activity was demonstrated against the immunising strain, the neo-epitope still dominated the immune response to the glycoconjugate precluding a functional response to wild-type strains. Re-examination of sera from our initial conjugation strategies revealed a direct correlation between the absence of neo-epitope recognition and the presence of functional activity, and we conclude that the titers of antibodies to the target oligosaccharide epitopes must not be diluted with antibodies to irrelevant epitopes in order to obtain a functional immune response.

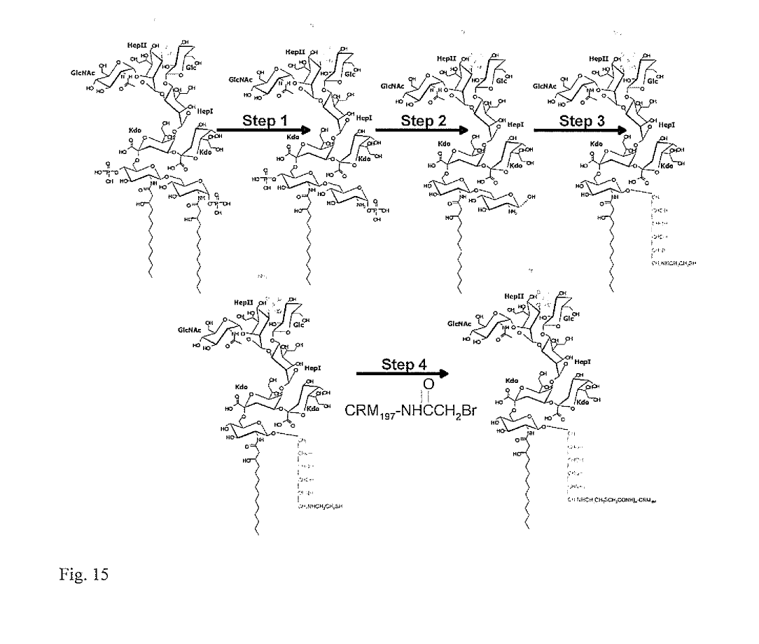

We subsequently designed alternate conjugation strategies in an attempt to avoid these problems. Both strategies involved utilizing the slime mould Dictyostelium discoideum (Dd) which produces esterases (FAE) and amidases (FAA) which specifically remove the fatty acids from the lipid A region without affecting the carbohydrate region of the molecule. In the environment Dd feeds on bacteria, engulfing the organism, removing the fatty acids on the lipid A of the LPS as its food source. Dd acts on LPS in a specific sequential manner initially utilising esterases to remove the O-linked fatty acids and then removing the two N-linked fatty acids in turn, utilising enzymes FAAI and FAAII (FIG. 14). In this way the immunologically important PEtn residue can be retained in the completely deacylated LPS molecule, a feat which was not possible using chemical methods. Our first strategy was a direct reductive amination conjugation to the Dictyostelium N-deacylated carbohydrate molecule without any linker molecule, akin to the approach we had adopted with O-deacylated that had proven successful previously. Our second strategy involved the linker cystamine so that sulphur chemistry that would not involve the PEtn residue could be used in the conjugation strategy (FIG. 15). Described herein is the chemistry of the production of these glycoconjugates and their immunology following immunisation of mice and rabbits.

### Characterisation of Dd Amidase Treated LPS-OH

The extent and specificity of de-N-acylation achieved during treatment with Dd amidases was effectively monitored by MS and NMR. MS/MS analysis clearly revealed the complete removal of one N-linked fatty acid and some partial removal of a second N-linked fatty acid. This was confirmed by a tandem mass spectrometry technique, which can specifically fragment selected ions from the primary mass spectrum. The nature of LPS-OH and derived molecules is such that fragmentation is enhanced between the lipid A region and the core oligosaccharide molecule, with the size of the fragmented lipid A region being indicated in the resulting mass spectrum. In this way one can compare the size of the lipid A region from intact LPS-OH to that of the product from LPS-OH exposure to the Dd/Ka suspension.

An intact LPS-OH molecule from strain galE fragments to give ions for the lipid A region of m/z 952⁻ and 475²⁻. These ions of m/z 952⁻ and 475²⁻ correspond to two glucosamine sugars, two phosphate groups and two N-linked fatty acid moieties.

However, MS/MS analysis of the doubly charged ion at m/z 1018.7²⁻ from the LPS-ONH molecule causes fragmentation to give a singly charged ion for the lipid A region of m/z 725″. This ion of m/z 725″ corresponds to two glucosamine sugars, two phosphate groups and one N-linked fatty acid moiety, thus illustrating that a N-linked fatty acid has been removed from the lipid A region of the LPS-OH following exposure to the Dd/Ka suspension. The core oligosaccharide is still completely intact as indicated by a singly charged ion at m/z 1311.6 that corresponds to a composition of 2 Kdo, 2Hep, PEtn, GlcNAc, Glc and the loss of one Kdo residue (due to the labile nature of the ketosidic bond in the MS fragmentation step) to give the singly charged ion at m/z 1091⁻. Proof that the N-linked fatty acid on the reducing glucosamine residue of lipid A had been removed was obtained by NMR experiments. A well-resolved ¹H-NMR spectrum was obtained consistent with a water-soluble molecule being produced following removal of at least one N-linked fatty acid. A 2D COSY spectrum identified the H-2 resonances. The upfield shift of the H-2 resonance of the glucosamine bearing the glycosidic phosphate to 3.4 ppm, was consistent with the removal of the N-linked fatty acid. A ¹³C-¹H HSQC experiment confirmed that this H-2 resonance was on a carbon residue attached to a nitrogen atom by virtue of its diagnostic ¹³C shift of ˜54 ppm. It can also be noted that signals indicative of the retention of the PEtn residue are still observed at intensities consistent with stoichiometric attachment. Therefore treatment with amidases from Dd has created a water soluble molecule, fully amenable to subsequent steps in the conjugation strategy, and the immunologically important PEtn residue has been retained.

Production of Cystamine Linker Glycoconjugates with Open-Chain Technology

Following alkaline phosphatase treatment cystamine was added to the carbohydrate and following reduction the product was characterized by mass spectrometry revealing that the linker had been efficiently incorporated into the carbohydrate molecule. The protein carrier CRM₁₉₇ was activated with approximately 17 bromo-acetyl groups being attached as deduced by MALDI-MS experiments. Activated CRM was conjugated to the cystamine coupled carbohydrate and unconjugated bromo-acetyl groups were capped.

Following conjugation the conjugate was purified as described in the material and methods and examined by SDS-PAGE. The migration pattern of the unmodified CRM, activated CRM and conjugate are all consistent with an increase in size of the molecule. The conjugate reacted with the carbohydrate specific mAb B5 in a Western blot.

### Immunogenicity of Glycoconjugates

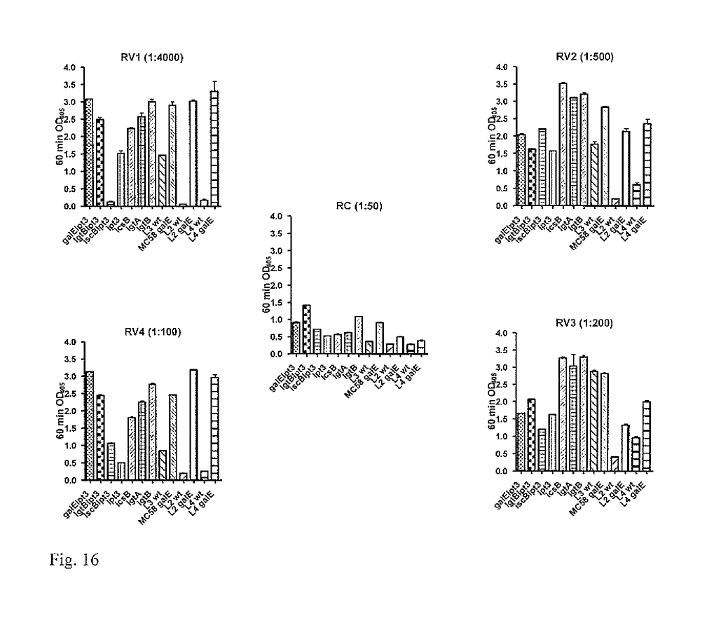

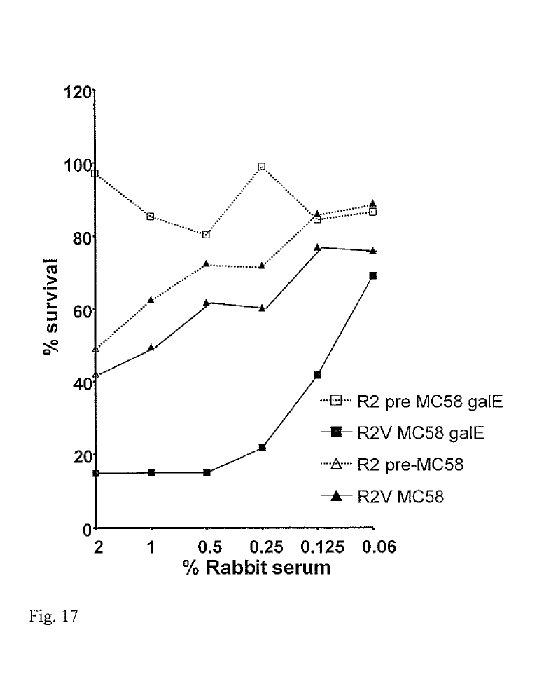

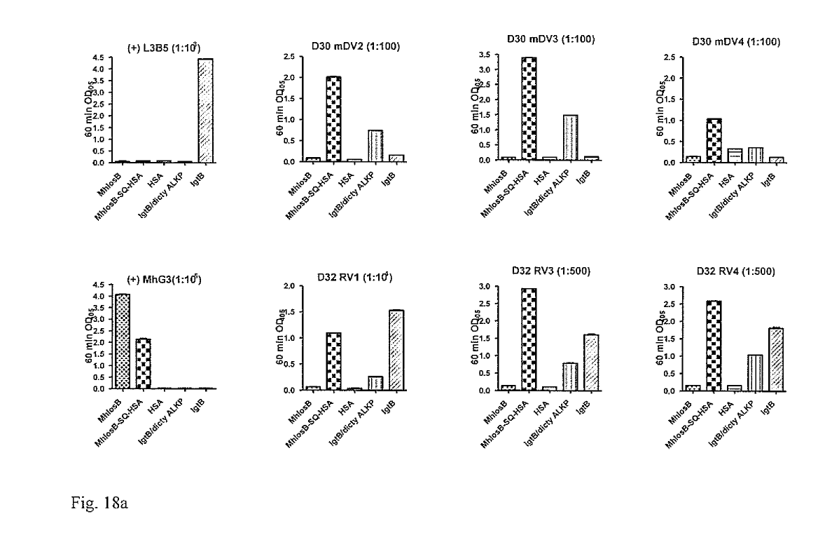

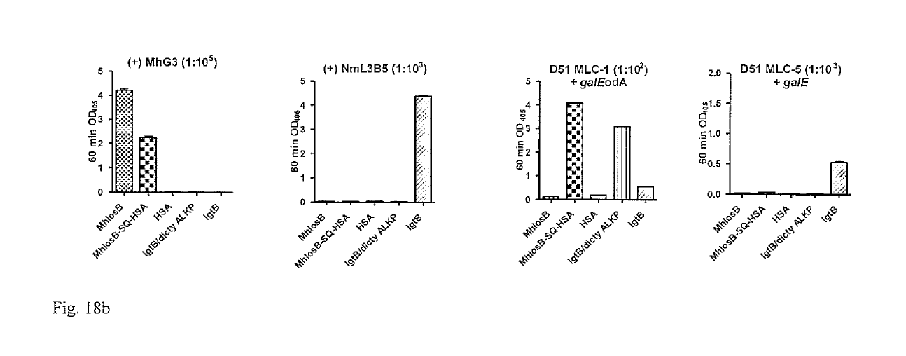

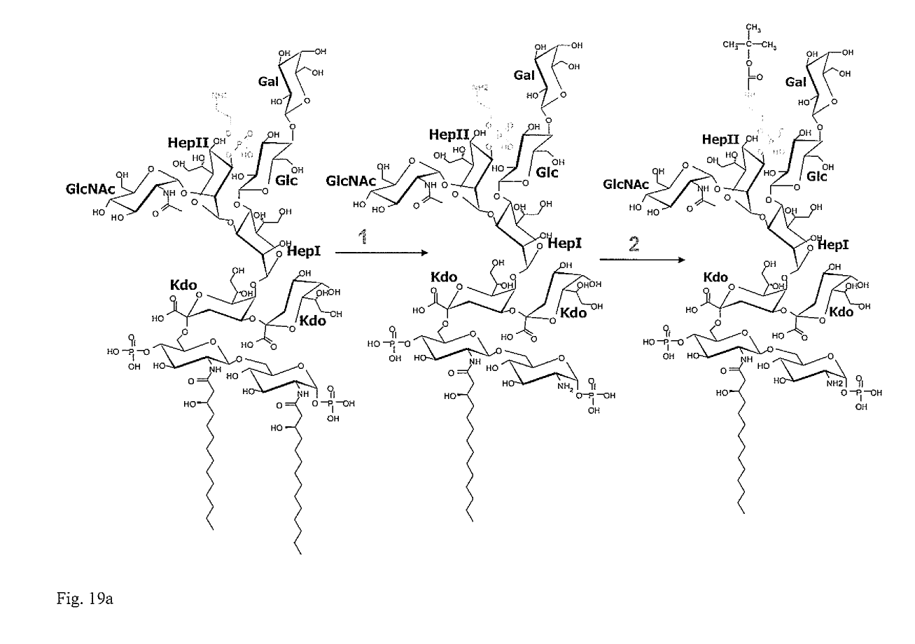

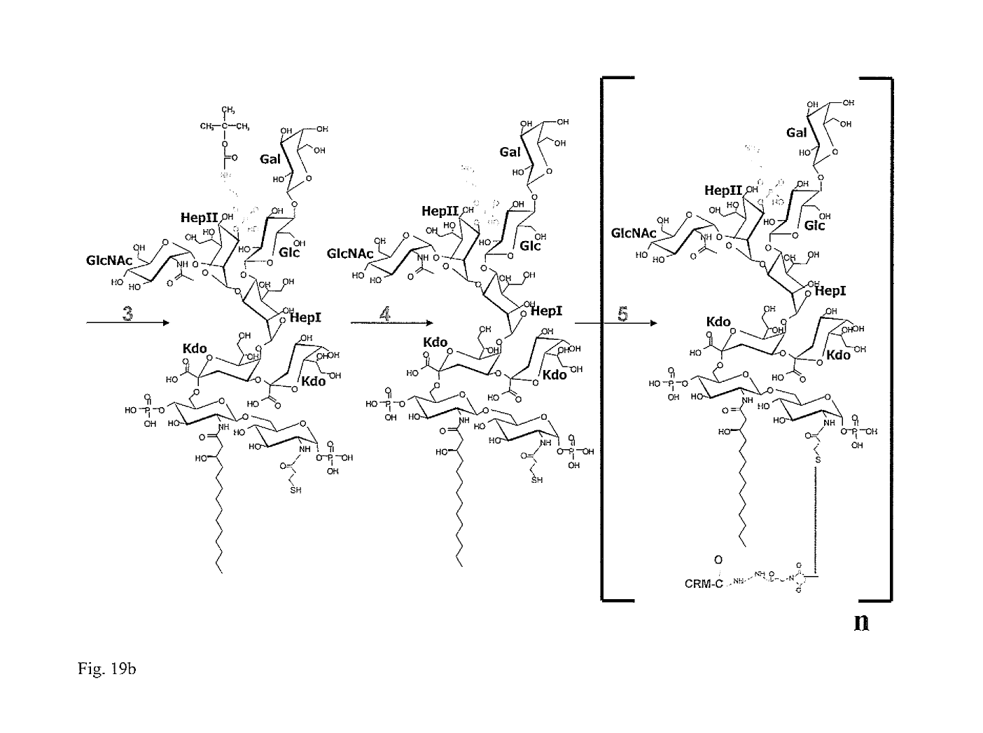

Mice and rabbits were immunized with a prime and boost strategy of 10 and 50□g of conjugated carbohydrate with Ribi and Freunds adjuvants respectively. Sera were examined for titer to the homologous LPS antigen by ELISA. None of the mice sera from the reductive amination or cystamine conjugates recognised the homologous LPS, whereas rabbit sera from the cystamine conjugate did. These rabbit's sera were subsequently tested for their ability to recognise a range of meningococcal LPSs (FIG. 16). The sera were broadly cross-reactive, recognizing wild-type fully extended glycoforms when PEtn was located at the 3-position of the distal heptose residue of the inner core (the same location as in the immunising antigen). Wild-type LPS glycoforms elaborating a PEtn residue at the 6-position were not recognised (truncated structures bearing a 6-PEtn were recognised, presumably due to a population of antibodies recognising the terminal glucose residue) reinforcing the specificity of the immune response. The rabbit sera were functional in bactericidal assays against the galE mutant but not against the wt strains (FIG. 17). This was attributed to the high proportion of the sera that was recognizing the open-chain neo-epitope, and this neo-epitope recognition was also observed as the exclusive response in the mice sera (FIG. 18). To determine that a neo-epitope was being recognised we used structurally diverse LPS from Mannheimia haemolytica mutant strain losB [20] and in a conjugated form to a different protein, HSA via a squarate and not cystamine linker, the only common feature therefore between the conjugate used for immunisation and that on the ELISA plate being the open chain reducing end glucosamine residue. MAbs L3B5 and MhG3 were utilised to confirm the presence of the various antigens on the ELISA plate. The three mice sera examined each recognised the Mh losB conjugate most strongly with no recognition of the Mh losB LPS and no to minimal recognition of the HSA protein, thus illustrating that the conjugate recognition is due to the only common feature shared by the immunising conjugate and the Mh losB conjugate, the open chain residue. The rabbit sera behaved similarly with the majority of the immune response directed to the Mh losB conjugate, but in the case of rabbits there was some recognition of the lgtB LPS which elaborates the target inner core structure. Although LPS molecules elaborating the target oligosaccharide were recognised following immunisation in rabbits with these conjugates, the presence of PEtn was not sufficient to overcome the immunodominance of the open-chain sugar created with this conjugation strategy and enable bactericidal activity against wild type strains. We therefore decided to examine the mice sera from the initial conjugation strategy where both PEtn and the open chain residue were present, but in some cases functional activity against wild-type strains was observed, for the estimation of the population of sera capable of recognising the neo-epitope (FIG. 18b). Interestingly, the neo-epitope was also observed with sera from the initial conjugates prepared via a direct reductive amination strategy, but significantly functional sera (MLC-5) did not contain any antibodies to the neo-epitope whereas antibodies to the neo-epitope were dominant in non-functional sera (MLC-1).

### Example 3

### Preparation of Conjugates with Reducing End in Cyclic Form: Neisseria meningitidis

The foregoing studies identified the potential of LPS-based vaccines to combat meningococcal disease, but failed to produce protective antibodies. These approaches did however identify the creation of a neo-epitope during the conjugation protocol which dominated the immune response precluding a satisfactory response to the target region. This neo-epitope was identified as the open-chain reducing glucosamine residue so-formed after removal of the glycosidic phosphate moiety. Described herein is a novel conjugation strategy that still targets the terminal glucosamine disaccharide as the point of attachment to the carrier protein, but with the retention of the cyclic nature of these residues. To achieve this goal we have further developed the use of amidases produced by the slime mould Dictyostelium discoideum. We targeted the amino functionality created by the amidase activity as the point of attachment for the carrier protein. In order to protect the amino functionality on the immunologically important phosphoethanolamine residue of the inner core oligosaccharide, we developed a novel blocking and unblocking strategy involving the use of t-butyl oxycarbonyl (Boc). Using a maleimide-thiol linker strategy, targeting lysine residues on the carrier protein we were unable to obtain sufficient loading of the carbohydrate molecules per protein carrier. However when we targeted the carboxyl residues of the protein carrier with a similar maleimide-thiol linker strategy we obtained a loading of ˜12-15 carbohydrates per protein. These ‘high-loading’ conjugates were immunised into mice and rabbits and we obtained broadly cross-reactive antisera following rabbit immunizations which were capable of recognising fully extended wild-type strains that elaborated a PEtn residue at the 3-position of the distal heptose residue of the inner core oligosaccharide. However we also observed a significant immune response to any free maleimide linkers on the protein surface.

An effective broadly cross-reactive vaccine to combat disease caused by the serogroup B meningococcus remains the main challenge for researchers in this field. Glycoconjugate vaccines based on the capsular polysaccharides are currently available and have proven successful in protecting against serogroups A, C, W-135 and Y. Unfortunately the serogroup B capsule consists of an α-2,8-sialic acid polymer which is poorly immunogenic, especially in infants as it mimics glyco-modifications on host neuronal cells. Therefore alternative vaccine antigens are being sought including modified capsular polysaccharide, outer membrane vesicles, attenuated vaccines, common antigens identified in Neisseria lactamica and outer membrane proteins identified from a reverse vaccinology approach. Some of these candidates; N. lactamica OMV, PorA and a genome derived pentavalent vaccine are in early phase I or phase II trials.

Our strategy is to use inner core LPS that has been shown to be conserved in the majority of NmB strains, accessible to antibodies and able to elicit functional Abs against NmB strains. In our next strategy the amino functionality created by removal of the N-linked fatty acid is targeted in the conjugation reaction, thus avoiding the need to create an aldehydro functionality via glycosidic phosphate removal and ring-opening. The competing amino functionality of the PEtn residue is selectively protected and de-protected. Glycoconjugates were produced that were still linked to the carrier protein via the reducing glucosamine residue, but most importantly without the creation of the immunodominant open-chain neo-epitope.

Lysine targeted conjugates were prepared. Carboxyl targeted conjugates were prepared according to the scheme illustrated in FIG. 19. Each step of the strategy was quality controlled by MS and or NMR as appropriate.

### Characterisation of Dd Amidase Treated LPS-OH.

The extent and specificity of de-N-acylation achieved during treatment with Dd amidases was effectively monitored by MS and NMR as described previously [8].

### Protection of PEtn Residue

Following de-N-acylation, the PEtn residue on the galE derived molecule was selectively protected and the product was characterised by mass spectrometry. MS analysis of the material before and after Boc protection illustrated that the molecule had increased in mass by 100 amu, consistent with the incorporation of one Boc moiety. Small amounts of glycoforms consistent with the incorporation of a second Boc group were observed. As evidenced by MS/MS analysis, the Boc protecting group was localised to the core oligosaccharide portion of the carbohydrate molecule as the mass of the de-N-acylated glucosamine disaccharide region (m/z 362.5²⁻, 725.7⁻) was consistent with no Bac incorporation and the mass of core OS (m/z 1192.0⁻) was consistent with the incorporation of a Boc molecule. MS/MS analysis on the two Boc containing molecule revealed that both the reducing glucosamine disaccharide (m/z 412.0²⁻, 825.3⁻) and the core OS (m/z 1192.0⁻) had increased by 100 amu. MS characterisation was also obtained for the Boc-protected molecule from the lgtA mutant.

### Attachment of Linker

When lysine groups were targeted we attached a maleimide containing linker to the galE molecule as detailed in the material and methods and characterised by MS. An increase in mass of 165 amu was observed, as evidenced by the identification of ions (m/z 575.2⁴⁻, 767.1³⁻, 1151.2²⁻) consistent with the incorporation of the maleimide linker, when compared to the molecule without linker incorporation. When carboxyl groups were targeted we attached either a thiol or a maleimide containing linker to the lgtA molecule as detailed in the material and methods and characterised by MS.

### De-Protection of PEtn Residue

Following linker incorporation the t-Boc blocking group was efficiently removed from the PEtn residue of the galE derivative by treatment with 20% TFA with no effect on the remainder of the molecule as characterised by MS. A decrease in mass of 100 amu was observed, as evidenced by the identification of ions (m/z 550.3⁴⁻, 733.8³⁻, 1101.3²⁻) consistent with the removal of the Boc protecting group, when compared to the Boc-protected molecule. Similar results were obtained to characterise the efficient de-protection of the lgtA derivative.

### Activation of Protein Carrier

Lysine groups of CRM₁₉₇ were activated with a thiol containing linker (DTSP) as described in the material and methods and characterised by MALDI-MS which revealed that ˜24 lysine residues had been activated as evidenced by a mass increase of ˜2 kDa (Table 1).

Carboxyl groups of CRM₁₉₇ were activated with either a maleimide containing linker (BMPH) or a thiol containing linker (ADH-SATP) as described in the material and methods and characterised by MALDI-MS which revealed that ˜30 carboxyl residues had been activated as evidenced by a mass increase of ˜5.5 kDa (Table 1).

### Characterisation of Conjugation Products

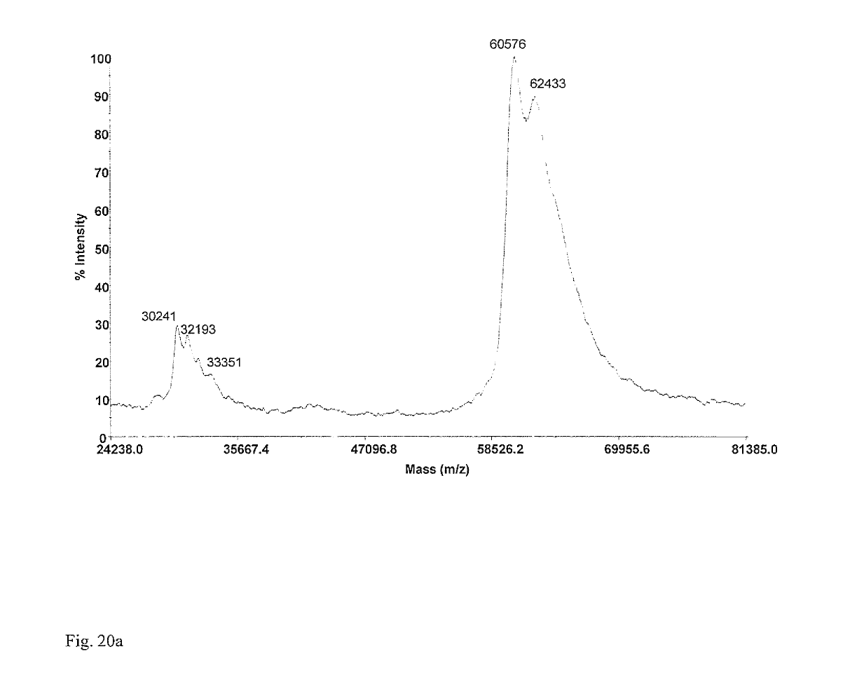

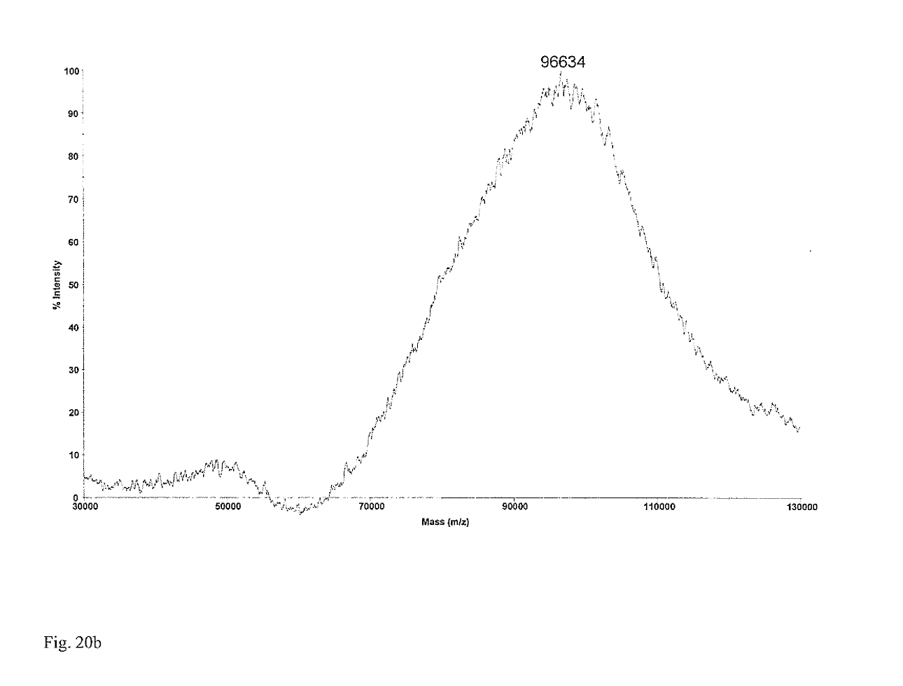

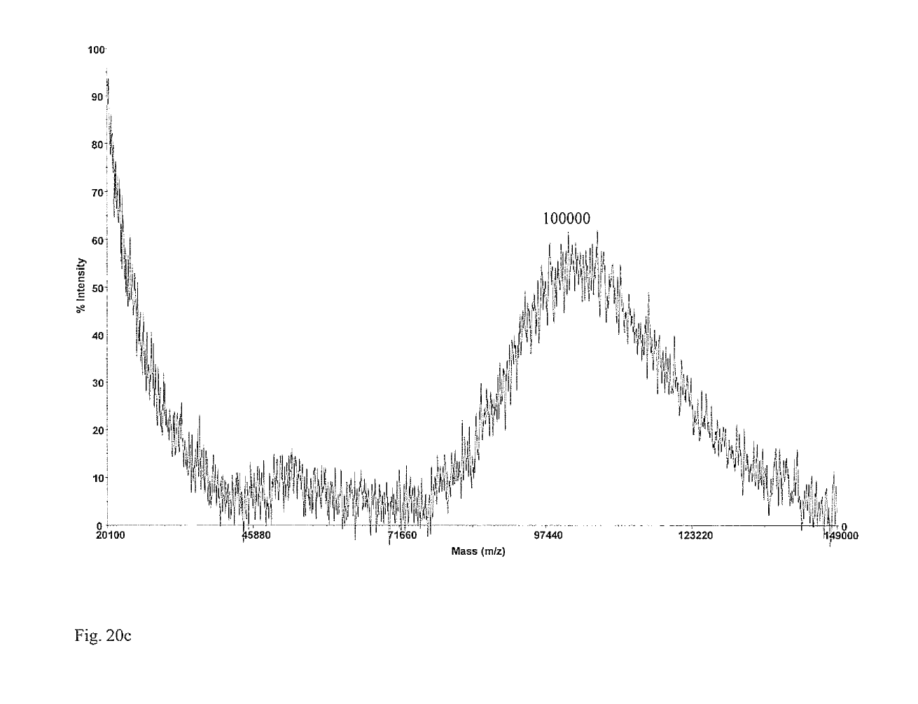

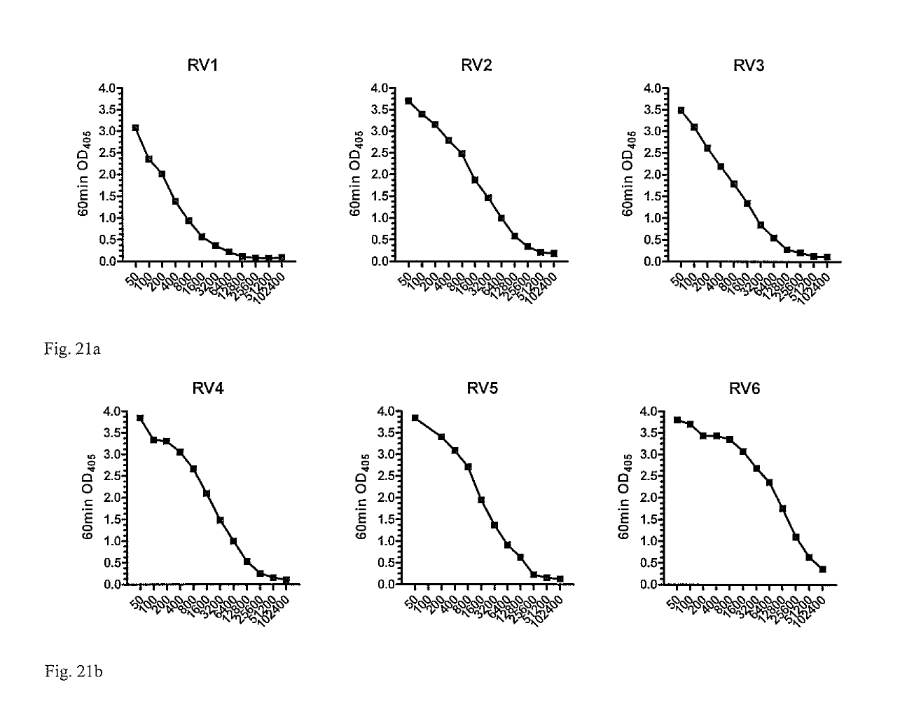

Activated CRM₁₉₇ was conjugated to the carbohydrate via the appropriate linker molecules as described in the material and methods. Conjugation products were purified as described and monitored by MALDI-MS (FIG. 20), SDS-PAGE and Western blotting. For the lysine linked conjugates the migration pattern on SDS-PAGE of the unmodified CRM₁₉₇, activated CRM₁₉₇ and conjugate are all consistent with a modest increase in size of the molecule. The conjugate reacted with the carbohydrate specific mAb B5 in a Western blot, confirming incorporation of some carbohydrate and MALDI-MS (FIG. 20a) revealed that perhaps only 1 or at best two carbohydrate molecules had been attached to the protein carrier as evidenced by a modest increase in mass of ˜2 kDa when compared to the activated carrier protein (Table 1). For the carboxyl linked conjugates with maleimide linkers on the protein carrier, the SDS-PAGE migration pattern of the conjugate was consistent with a significant increase in the size of the molecule and Western blotting revealed strong recognition with mAb B5. MALDI-MS analysis revealed that approximately 14 carbohydrates had been incorporated per protein carrier (FIG. 20b) as evidenced by the significant increase in mass of ˜33 kDa when compared to the activated carrier protein (Table 1). Similar MALDI-MS data was obtained for the carboxyl linked conjugates with thiol linkers on the protein carrier, suggesting ˜16 carbohydrate molecules had been attached per carrier protein (FIG. 20c, Table 1). This conjugate also showed considerably slower migration than the activated protein carrier on SDS-PAGE and reacted with mAb B5 in a Western blot.

Immunogenicity of glycoconjugates

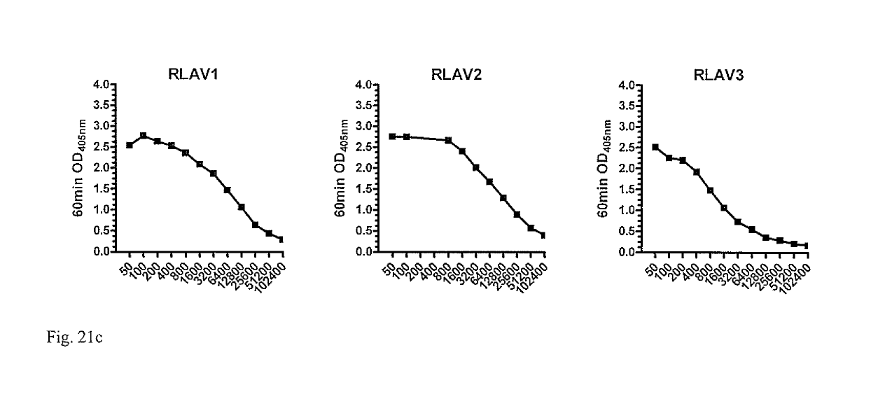

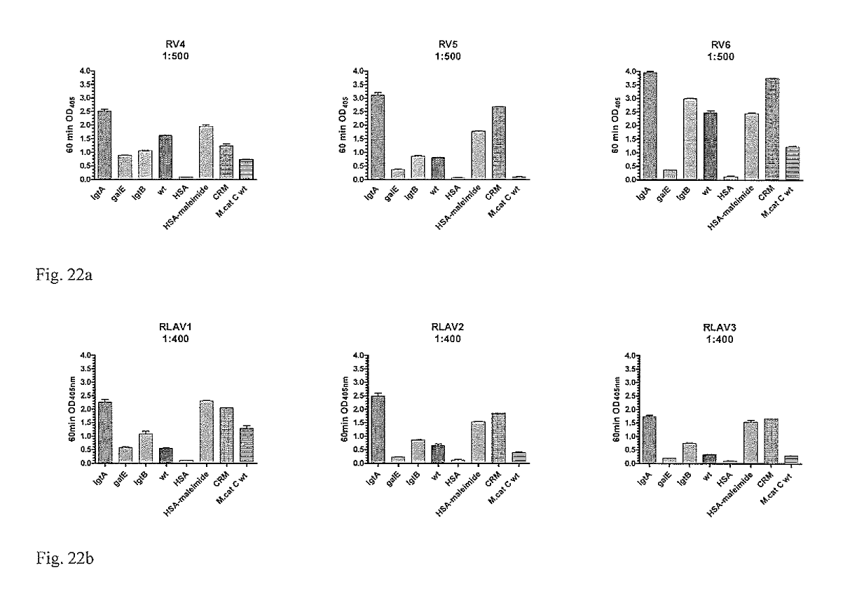

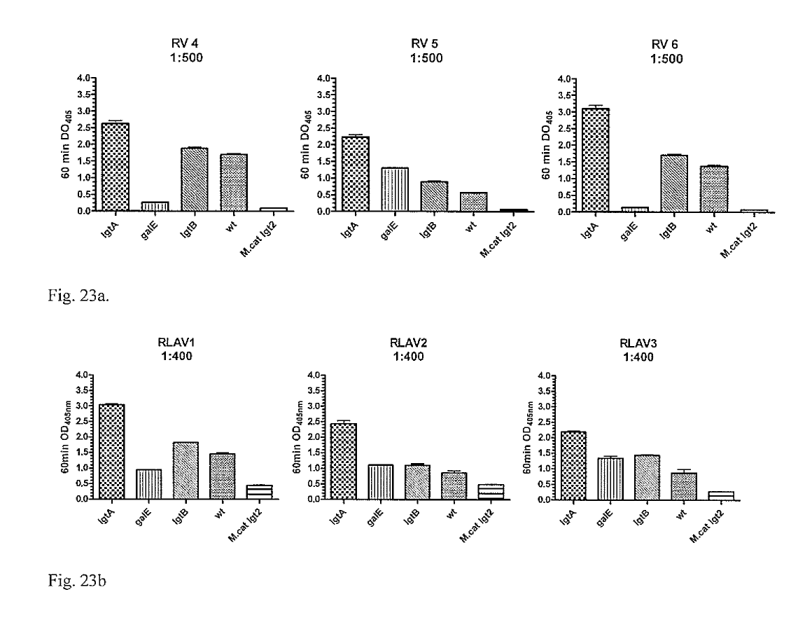

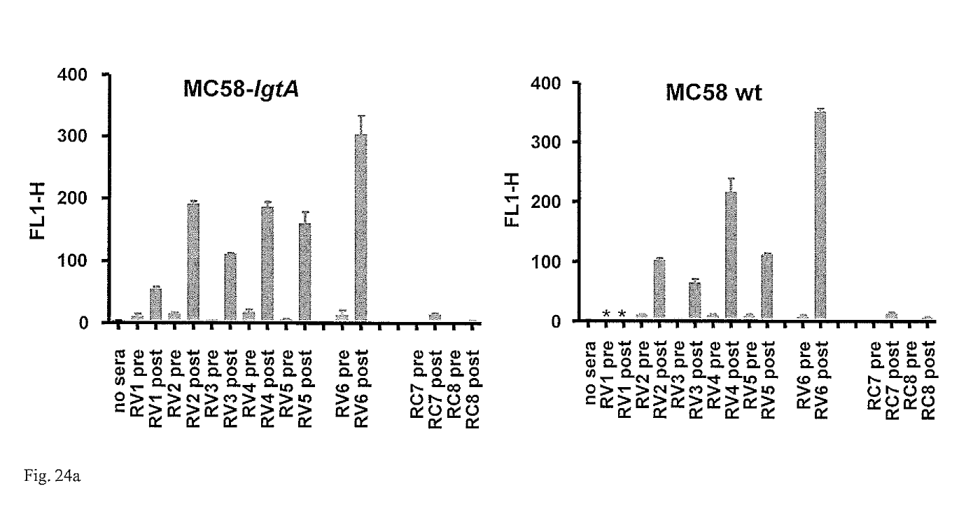

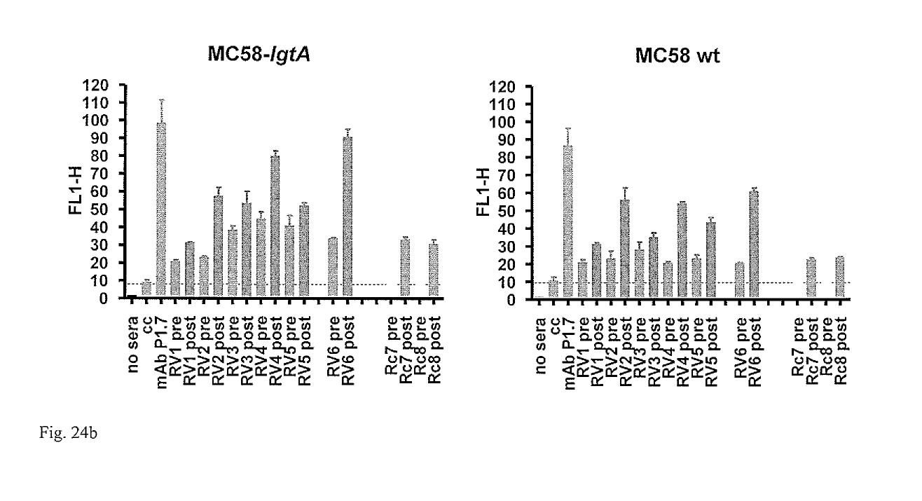

Due to insufficient carbohydrate loading the CRM-DTSP-galE conjugate was not used in immunisation studies. Mice and rabbits were immunized, based on conjugated carbohydrate amounts, with a prime and boost strategy of 2, 5 and 10, and 25 and 50 μg of the CRM-BMPH-lgtA conjugate with Ribi and Freunds adjuvants respectively, and only rabbits at 28 ug of conjugated carbohydrate per immunisation with the CRM-ADH-SATP-lgtA conjugate with Freunds adjuvant. Rabbit sera were initially titrated against the homologous antigen, Nm MC58-lgtA LPS which revealed good titers for the sera from rabbits that received the 50 ug CRM-BMPH-lgtA and 28 ug CRM-ADH-SATP-IgfA conjugates, with rabbits that received the 25 ug CRM-BMPH-lgtA conjugate not responding to the lgtA LPS antigen quite as well (FIG. 21). These sera were subsequently tested for cross reactivity against a range of Nm strains and also tested against HSA with maleimide attached, akin to the activated carrier protein in order to ascertain the level of immune response to the maleimide functionality (FIG. 22). This revealed a significant, although not immunodominant response to the maleimide functionality from both style lgtA conjugates, as there was also a reasonable profile of cross reactivity with the homologous (lgtA) and more extended (lgtB and wt) LPSs being recognised (FIG. 22). The terminal galactose residue was immunologically the most important feature of the immunogen as more truncated (galE) LPS was not recognised (FIG. 22). Mice sera revealed a quite different cross-reactivity profile (data not shown) with only O-deacylated LPS being recognised and no significant response to the maleimide moiety. This result suggests that in mice the Kdo-Kdo-Lipid A OH region of the molecule is immunodominant and for this reason the mice sera were not studied further. Rabbit sera were also capable of recognising a variety of meningococcal whole cells when compared to the control sera and the recognition was shown to be specific as control cells from the structurally non-related Moraxella catarrhalis were not recognised (FIG. 23). Rabbit sera were then examined for their ability to facilitate complement mediated bactericidal killing of meningococcal cells. Post-immune sera were compared to pre-immune controls from the same rabbits. Clear evidence of bactericidal killing was only observed with RV6 of the CRM-BMPH-lgtA conjugate immunised animals and only against the homologous lgtA mutant and not the wild type strains (Table 2). This is consistent with the high titers of these rabbit sera. However, clear evidence of bactericidal killing was observed with each rabbit that had received the CRM-ADH-SATP-lgtA conjugate, with a hint of killing of the wild-type H44176 strain also observed with sera derived from rabbits immunised with this conjugate (Table 2). We therefore examined if the rabbit polyclonal sera derived from the CRM-BMPH-lgtA conjugate immunisations were able to surface label and facilitate complement deposition of mutant and wild type meningococcal cells, two key steps essential to enable bactericidal killing. As illustrated in FIG. 24, the rabbit polyclonal sera derived from the CRM-BMPH-lgtA conjugate immunisations were capable of surface labelling (FIG. 24a) and facilitating complement deposition (FIG. 24b) on both mutant and wild-type cells.

This study has described a novel conjugation strategy that has facilitated the preparation of conjugates with a high loading of carbohydrate molecules (12-15) per carrier protein. The loading achieved here can be qualitatively and quantitatively characterised by SDS-PAGE I Western and MALDI MS techniques respectively. The high loading achieved with this methodology is a significant improvement over previous conjugate preparations where evidence of perhaps at best 2-3 carbohydrate molecules per carrier protein was obtained. This improvement is evidently due to targeting of carboxyl groups rather than amino functionalities on the carrier protein. The carrier protein used in these studies, CRM₁₉₇, contains 63 carboxyl and 39 amino functionalities, so although there are more carboxyl moieties available, it is unlikely that the increase in loading is simply due to the number of functionalities available, but perhaps the accessibility of the targeted functionalities. Clearly in this study using the same linker chemistries with maleimide and thiol groups we struggled to attach 2 carbohydrates per CRM₁₉₇ targeting lysine residues, whereas 12-15 carbohydrates were attached targeting carboxyl groups. It was anticipated that this significant improvement in loading would lead to improved titers to the carbohydrate conjugated to the carrier, and this did indeed appear to be the case. In employing the conjugation strategy described here, we did successfully avoid the previously identified immunodominant open chain epitope by utilising the amino functionality created at the reducing end of the carbohydrate by enzyme treatment with an amidase from Dictyostelium discoideum. This amino group could then be targeted as the site of conjugation whilst retaining the cyclic nature of the terminal glucosamine residue. However this then presented the challenge of selectively protecting the amino functionality of the immunologically important PEtn residue in the inner core OS. We achieved this by utilising a simple blocking strategy which preferentially attached to the inner core PEtn amino when ratios of blocking agent and substrate were carefully controlled. The remaining amino functionality was now uniquely available for reaction with the linker molecule, and the blocking group was then subsequently removed, thus directing the location of conjugation between the carbohydrate and carrier protein. This elegant chemical methodology has enabled us to construct conjugates with high carbohydrate loading whilst maintaining conjugation via the lipid A region of the molecule but without introducing an open chain epitope which we have previously shown to be immunodominant, nor modifying the immunologically important PEtn inner core OS residue. High titers were observed with the conjugate derived rabbit sera to the homologous lgtA antigen and good cross-reactivity was found to further extended outer core oligosaccharides from mutant and wild type meningococcal strains. There was evidence that a high proportion of the immune response was to the galactose residue at the tip of the core OS of the immunising lgtA antigen, as LPS not elaborating that terminal sugar, e.g. galE mutants were not recognised.

There did appear to be a correlation between serum titers and functionality, as clear bactericidal activity was obtained against the homologous antigen using sera from RV6 which was the best responder from the CRM-BMPH-lgtA conjugate immunised rabbits and from each of the CRM-ADH-SATP-lgtA conjugate immunised rabbits. In terms of the immunodominance of the linker, in the CRM-BMPH style conjugates, it appears that non-conjugated maleimide linker remaining upon the carrier protein, is more deleterious to the immune response to the target core oligosaccharide epitope than in the CRM-ADH-SATP style conjugates, where only conjugated maleimide linker will form part of the immunising conjugate, and underivatized linker will take the form thiol moieties which are perhaps less immunodominant. Coupled to this we have the mild immunodominance of the terminal galactose residue and it is plausible that titers to our target region were not sufficiently high to facilitate bactericidal killing of wild-type meningococcal strains consistently.

We were however encouraged that the present conjugates elicited specific inner core Abs that could bind to the cell surface of meningococci and demonstrated cross-reactivity to different inner core epitopes of both wt meningococci and mutants elaborating truncated LPS glycoforms, and furthermore, even the CRM-BMPH style conjugates were capable of mediating complement deposition, against wt strains. It seems likely that the concentration and/or avidity of the antibodies to wt strains were insufficient to result in bactericidal activity and perhaps if the competition with other epitopes is able to be reduced in order to favour our target core epitopes, bactericidal killing would result. Our future studies will focus on the CRM-ADH-SATP style conjugates with antigens that do not elaborate the terminal galactose residue.

### Example 4

### Preparation of Conjugates with Reducing End in Cyclic Form: Haemophilus influenzae

Conjugates were prepared from LPS oligosaccharides derived from NTHi strains using an approach that preserves the integrity of inner-core PEtn groups. We initially used NTHi strain 1003 lic1 IpsA (FIG. 5), which elaborates a PEtn residue on the minimum truncated structure which we have found to be present in all strains of NTHi examined, as the LPS substrate for conjugation experiments.

Immune responses were evaluated in mice and chinchillas. Chinchilla challenge will be carried out on those immunized animals showing high titres of functional induced antibody.

O-deacylated LPS (LPS-OH) from Hi strain 1003 lid IpsA was modified in order to prepare a glycoconjugate elaborating an inner core PEtn moiety according to methodologies we have developed for meningococcal vaccines (see above FIG. 19).

### Animal Studies

Mice and chinchillas have been immunised with the resulting conjugate and the derived sera were examined for titers and cross-reactivity.

### Mice Sera

Mice received a conjugate with an average of 13 carbohydrates attached as determined by MALDI-MS (Table 1). The final bleed sera was titrated against the LPS—OH and LPS from 1003 lic1 lpsA

Mice sera MAV1 and MBV4 were the only two that were capable of recognising LPS and these sera were subsequently tested for cross reactivity against a range of Hi strains and also tested against HSA with maleimide attached, akin to the activated carrier protein in order to ascertain the level of immune response to the maleimide functionality. This revealed no cross reactivity with other Hi strains, but there was a significant, immunodominant response to the maleimide functionality.

### Chinchilla Sera

Chinchillas received a conjugate with an average of 13 carbohydrates attached. The final bleed sera were titrated against the LPS-OH from 1003 lic1 lpsA.

These sera were subsequently tested for cross reactivity against a range of Hi strains and also tested against HSA with maleimide attached, akin to the activated carrier protein in order to ascertain the level of immune response to the maleimide functionality. This revealed no cross reactivity with other Hi strains, but there was a significant, immunodominant response to the maleimide functionality.

The immunological data from both mice and chinchillas has clearly indicated the immunodominant nature of the maleimide functionality. Even though high carbohydrate loading has been achieved by this methodology, the immunodominance of the maleimide group is precluding a strong immune response to the target core OS region. We therefore performed an alternate strategy, maintaining conjugation via —COOH groups on the protein, but replacing the maleimide functionality on the protein with a thiol moiety.

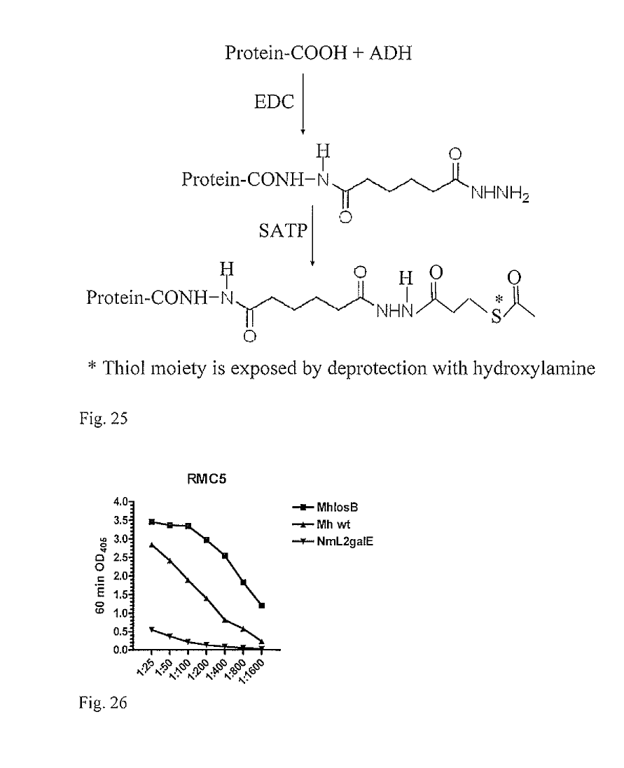

Thiol groups are incorporated via the EDC reaction at carboxyl groups of the carrier protein via a two step reaction (FIG. 25), and the thiol groups are subsequently reacted with maleimide groups of the carbohydrate in order to prepare the glycoconjugate.

MALDI-MS analysis (Table 1) revealed that approximately 25 carboxylic acid groups had been derivatized with thiol functionalities, thereby engineering sufficient locations on the carrier protein to facilitate high loading of carbohydrate molecules.

The activated protein was reacted with Haemophilus influenzae lic1 lpsA derivatized LPS via a maleimide linker (Sulfo-GMBS) that had been attached to the derivatized LPS molecule. The conjugate appeared to be around 100 kDa as determined by MALDI-MS (Table 1) and SDS-PAGE, followed by a Western blot that illustrated the carbohydrate was still conformed appropriately as it was recognised by a carbohydrate specific antibody.

Insufficient conjugate was available to immunise chinchillas, and the immune response in mice suggested the immunodominance of the linker portion of the glycoconjugate.

### Example 5

### Preparation of Conjugates with Reducing End in Cyclic Form: Mannheimia haemolytica

We used Mannheimia haemolytica (Mh) strain losB (FIG. 7), which elaborates a conserved truncated structure which we have found to be present in all strains of veterinary pathogens (Mh, Actinobacillus pleuropneumoniae (Ap) and Pasteurella multocida (Pm)) examined to date, as the LPS substrate for conjugation experiments.

The carbohydrate molecule was derivatized in the same way as for the previous examples.

Briefly, the O-deacylated LPS was treated with the isolated amidase activity from Dictyostelium discoideum and the maleimide linker was directly attached to this molecule.

The MS spectrum revealed the efficient removal of the N-linked fatty acids as evidenced by doubly and triply charged ions at m/z 906²⁻ and 604³⁻ and 946²⁻ and 631³⁻ that correspond to a composition of 2GlcN, Kdo, 4Hep, 2Glc and either two or three phosphate groups. The MS spectrum revealed the efficient incorporation of the maleimide linker as evidenced by doubly and triply charged ions at m/z 988²⁻ and 659³⁻ and 1028²⁻ and 685³⁻. The carrier protein was maltose binding protein (MBP), which was activated with the thiol linker SAT(PEO)₄. The native protein, the activated protein and the conjugate were all characterised by SDS-PAGE and MALDI.

The MALDI-MS data indicated that 3,000/307=10 SAT(PEO)₄ groups had been added to each MBP.

The MALDI-MS data indicated that 10,600 12,000=5 carbohydrates had been added to each MBP. This loading was lower than the 13 or 14 carbohydrates attached when targeting carboxyl residues as described above, but does illustrate the utility of the Dictyostelium enzymes to produce a carbohydrate molecule that can be conjugated from another bacterial species.

As a result of the relatively low carbohydrate loading, rabbit sera (trial and final bleeds) derived from immunisation with this conjugate was of low titer to the carbohydrate component.

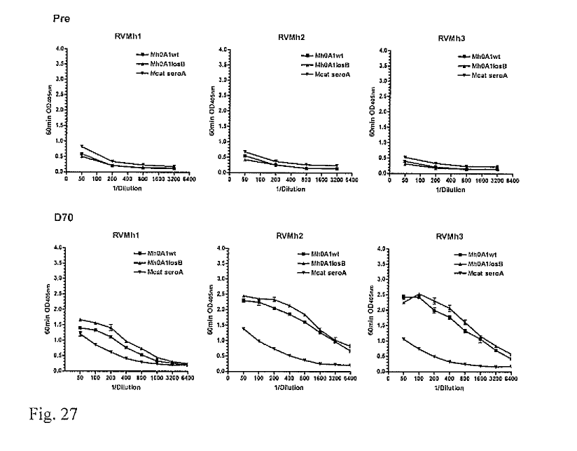

Nevertheless, sera from rabbit 5 was able to recognise whole cells of both the immunising strain and the parent strain, and the specificity was illustrated by an absence of recognition with an unrelated meningococcal strain (FIG. 26). In order to improve carbohydrate loading, a conjugates of Mh/osB was prepared by the carboxyl targeting methodology.

The CRM carrier protein was activated as described above and monitored by MALDI-MS at each step (Table 1). This revealed that ˜27 ADH moieties were added in the initial step, 21 SATP moieties were then added and the thiol functionality exposed, prior to conjugation which resulted in 10 carbohydrate moieties attached per protein.

The resulting conjugate was also examined by SDS-PAGE and Western blotting which corroborated the MALDI data and confirmed that the carbohydrate molecule was still appropriately conformed as it was recognised by a carbohydrate specific antibody.

The conjugate was used to immunise 3 rabbits based on 50 ug of conjugated carbohydrate per injection, using a prime and two boost strategy. 14 days after the final immunisation sera was obtained and examined for titer, cross-reactivity to LPS and ability to recognise whole cells (FIG. 27). Titers were somewhat low, but the sera from 2 of the 3 immunised animals were able to specifically recognise whole cells of Mannheimia haemolytica losB mutant and wt, and did not recognise whole cells from an irrelevant species Moraxella catarrhalis when compared to the pre-immune sera (FIG. 27).

### Example 6

### Preparation of Conjugates with Reducing End in Cyclic Form: Moraxella catarrhalis

We used Moraxella catarrhalis (Mc) serotype A mutants lgt2 and lgt2/lgt4 (FIG. 6), which elaborate conserved truncated structures which we have found to be present in all strains of Mc so far examined as the carbohydrate moieties to conjugate to the —COOH groups on CRM or other suitable carrier by methods described in other examples. Preliminary data had indicated that antibodies raised to the lgt2 LPS structure were capable of broad cross-reactivity amongst the three Mc serotypes and were functional in a bactericidal assay. Data had also revealed a potential for immunodominance of the terminal GlcNAc residue in the lgt2 mutant structure and thus a parallel strategy utilising a lgt2/lgt4 double mutant, lacking the terminal GlcNAc residue was also pursued.

A LPS-ONH-CRM₁₉₇ conjugate was prepared by conjugation to activated carboxyl groups of CRM₁₉₇ using maleimide-thiol linker chemistry, that involved initial steps to de-O- and de-N-acylate the lipid A region of the LPS molecule add the thiol linker to the unique amino functionality created by the de-N-acylation and conjugate to the carboxyl activated protein carrier.

The glycoconjugate was characterised by MALDI-MS (Table 1), SDS-PAGE and Western blotting revealing that approximately 11 carbohydrate molecules had been attached per carrier protein and that the carbohydrate was still conformed appropriately as it was recognised by mAb MC2-1.

### Immunisation and Conjugate Sera Cross-Reactivity

The immunogenicity of the conjugate was evaluated by immunization of rabbits with the prepared conjugate compared to the control sample (non-conjugated mixture of the LOS-OH with CRM₁₉₇ protein). IgG serum elicited against the conjugate showed the highest titer against the homologous lgt2 LOS of serotype A, followed by the wild type LOSs of the parent serotype A and serotype C.

The sera were also tested for their ability to recognise LPS and whole cells of serotypes A, B & C of Moraxella catarrhalis.

All sera did not recognise the irrelevant meningococcal cells, and the control sera were also negative. Sera from rabbit 1 recognised the lgt2 and serotype A strains the best, but also recognised serotype B and C cells well. Sera from rabbit 2 recognised the lgt2 and serotype A strains the best, but did not recognise serotype B and C cells. Sera from rabbit 2 only recognised the lgt2 strain.

The sera were then examined for their ability to facilitate bactericidal activity and were shown to effectively kill the homologous serotype A lgt2 mutant strain, but not the corresponding serotype A wild-type strain (Table 3)

A further LPS-ONH-CRM₁₉₇ conjugate was prepared by conjugation to activated carboxyl groups of CRM₁₉₇ using maleimide-thiol linker chemistry, that involved initial steps to de-O- and de-N-acylate the lipid A region of the lgt2/lgt4 LPS molecule addition of the thiol linker to the unique amino functionality created by the de-N-acylation and conjugation to the carboxyl activated protein carrier as described above. A further LPS-KOH-CRM₁₉₇ conjugate was prepared as above except that the lgt2/lgt4 LPS molecule was fully deacylated following treatment with KOH. The same conjugation protocol was applied from the linker chemistry onwards as described above.

The glycoconjugates were characterised by MALDI-MS, SDS-PAGE and Western blotting revealing that approximately 18 carbohydrate molecules and 9 carbohydrate molecules had been attached per carrier protein to the —ONH and —KOH conjugates respectively (Table 1). The carbohydrate was still conformed appropriately in both conjugates as it was recognised by mAb MC2-1.

### Immunisations and Conjugate Sera Cross-Reactivity

The immunogenicity of the conjugates was evaluated by immunization of rabbits with the prepared conjugates compared to the control samples (non-conjugated mixture of the LOS-OH with CRM₁₉₇ protein). IgG serum elicited against the —KOH conjugate showed the highest titer against the homologous lgt2/lgt4 LOS of serotype A, whilst serum elicited against the —ONH conjugate also good titers against the homologous lgt2/lgt4 LOS.

The sera were also tested for their ability to recognise LPS and whole cells of serotypes A, B & C of Moraxella catarrhalis and generally recognised wt serotypes A and B LPS better than serotype C. The sera were also able to recognise whole cells of serotypes A, B & C of Moraxella catarrhalis and had a preference for Moraxella cells when compared to irrelevant meningococcal cells, and the control sera were also negative. The sera were then examined for their ability to facilitate bactericidal activity and were shown to effectively kill the homologous serotype A lgt2/lgt4 mutant strain, and evidence of killing of the corresponding serotype A wild-type strain (Table 4).

### Example 7

### Preparation of Conjugates with Reducing End in Cyclic Form: Neisseria meningitidis

The Neisseria meningitidis galE LPS (FIG. 4) was prepared as described above. The CRM carrier protein was activated as described above and monitored by MALDI-MS at each step (Table 1). This revealed that ˜25 ADH moieties were added in the initial step, 18 SATP moieties were then added and the thiol functionality exposed, prior to conjugation which resulted in 17 carbohydrate moieties attached per protein.

The resulting conjugate was also examined by SDS-PAGE and Western blotting which corroborated the MALDI data and confirmed that the carbohydrate molecule was still appropriately conformed as it was recognised by a carbohydrate specific antibody (mAb B5).

The conjugate was used to immunise 6 rabbits based on 25 and 50 ug of conjugated carbohydrate per injection, using a prime and two boost strategy. 14 days after the final immunisation sera was obtained and examined for titer, cross-reactivity to LPS and ability to recognise whole cells. Titers were high from three rabbits, and these sera were broadly cross reactive against all further extended structures of meningococcal LPS including lgtA, lgtB and wt LPS.

The sera from these 3 immunised animals were able to specifically recognise whole cells of Neisseria meningitidis mutants lgtA, lgtB and the homologous galE and wt, and did not recognise whole cells from an irrelevant species Moraxella catarrhalis.

Rabbit sera were then examined for their ability to facilitate complement mediated bactericidal killing of meningococcal cells. Post-immune sera were compared to pre-immune controls from the same rabbits. Clear evidence of bactericidal killing was observed against the homologous galE mutant with a hint of activity against wild type strains (Table 5).

### Example 8

### Preparation of Conjugates with Reducing End in Cyclic Form: Neisseria meningitidis

The Neisseria meningitidis lgtB LPS (FIG. 4) was prepared as described above. The CRM carrier protein was activated as described above and monitored by MALDI-MS at each step (Table 1). This revealed that ˜21 ADH moieties were added in the initial step, 21 SATP moieties were then added and the thiol functionality exposed, prior to conjugation which resulted in 16 carbohydrate moieties attached per protein.

The resulting conjugate was also examined by SDS-PAGE and Western blotting which corroborated the MALDI data and confirmed that the carbohydrate molecule was still appropriately conformed as it was recognised by a carbohydrate specific antibody (mAb B5).

The conjugate was used to immunise 5 rabbits based on 25 and 50 ug of conjugated carbohydrate per injection, using a prime and two boost strategy. 14 days after the final immunisation sera was obtained and examined for titer, cross-reactivity to LPS and ability to recognise whole cells. Titers were highest from the three rabbits that received the 50 ug immunisations, and cross reactive against homologous lgtB and wt LPS, but not more truncated LPS. The sera from these immunised animals were able to specifically recognise whole cells of Neisseria meningitidis mutants lgtA, lgtB and the homologous galE and wt, and specificity was clear at higher dilutions as whole cells from an irrelevant species Moraxella catarrhalis were not recognised (RBV3 & 5). Rabbit sera were then examined for their ability to facilitate complement mediated bactericidal killing of meningococcal cells. Post-immune sera were compared to pre-immune controls from the same rabbits. No evidence of bactericidal killing could be confirmed against the homologous lgtB mutant as it was found that this mutant is sensitive to complement alone. A hint of activity was observed against wild type strain H44/76 with RBV3 sera (Table 6).

### Example 9

### Preparation of Conjugates with Reducing End in Cyclic Form: Neisseria meningitidis

The Neisseria meningitidis icsB LPS was prepared as described above. The CRM carrier protein was activated as described above and monitored by MALDI-MS at each step (Table 1). This revealed that ˜29 ADH moieties were added in the initial step, 21 SATP moieties were then added and the thiol functionality exposed, prior to conjugation which resulted in 9 carbohydrate moieties attached per protein.

The resulting conjugate was also examined by SDS-PAGE and Western blotting which corroborated the MALDI data and confirmed that the carbohydrate molecule was still appropriately conformed as it was recognised by a carbohydrate specific antibody (mAb B5).

The conjugate was used to immunise 3 rabbits based on 50 ug of conjugated carbohydrate per injection, using a prime and two boost strategy. 14 days after the second immunisation, sera was obtained and examined for titer, cross-reactivity to LPS and ability to recognise whole cells. Titers were high from one rabbit, and the sera was able to specifically recognise whole cells of Neisseria meningitidis mutants lgtA, lgtB, galE and the homologous icsB and wt, and did not recognise whole cells from an irrelevant species Moraxella catarrhalis, compared to control sera that was obtained from a rabbit that received carbohydrate mixed but not conjugated to the carrier protein CRM.

Rabbit sera were then examined for their ability to facilitate complement mediated bactericidal killing of meningococcal cells. Post-immune sera were compared to pre-immune controls from the same rabbit. Clear evidence of bactericidal killing was observed against the homologous icsB mutant with a hint of activity against wild type strain H44/76 (Table 7).

While the preferred embodiments of the invention have been described above, it will be recognized and understood that various modifications may be made therein, and the appended claims are intended to cover all such modifications which may fall within the spirit and scope of the invention.

TABLE 1
MALDI-MS analyses of CRM₁₉₇, activated CRM₁₉₇ and glycoconjugates

| | Conjugate | | | | | | | |
|---|---|---|---|---|---|---|---|---|
| | | CRM- | CRM-BMPH | CRM-ADH | CRM- | CRM-DTSP- | CRM-BMPH- | CRM-ADH- |
| | | DTSP | via | via | ADH-SATP | galE | CHO | SATP-CHO |
| MALDI-MS data (kDa) | CRM | via lysine | carboxyl | carboxyl | via carboxyl | via lysine | via carboxyl | via carboxyl |
| Neisseria meningitidis | | | | | | | | |
| CRM-ADH-SATP-galE | 58.3 | — | — | 62.2 | 64.6 | — | — | 100.0 |
| | | | | => 25 | => 18 | | | => 17 |
| | | | | carboxyls | ADHs | | | carbohydrates |
| | | | | activated | activated | | | attached |
| CRM-DTSP-galE | 58.3 | 60.4 | — | — | — | 60.6, 62.4 | — | — |
| | | => 24 | | | | => 0-1 | | |
| | | lysines | | | | carbohydrate | | |
| | | activated | | | | attached | | |
| CRM-BMPH-lgtA | 58.3 | — | 63.8 | — | — | — | 96.6 | — |
| | | | => 29 | | | | => 14 | |
| | | | carboxyls | | | | carbohydrates | |
| | | | activated | | | | attached | |
| CRM-ADH-SATP-lgtA | 58.3 | — | — | 62.2 | 64.8 | — | — | 100.0 |
| | | | | => 25 | => 20 | | | => 16 |
| | | | | carboxyls | ADHs | | | carbohydrates |
| | | | | activated | activated | | | attached |
| CRM-ADH-SATP-lgtB | 58.3 | — | — | 61.8 | 65.0 | — | — | 105.0 |
| | | | | => 21 | => 21 | | | => 16 |
| | | | | carboxyls | ADHs | | | carbohydrates |
| | | | | activated | activated | | | attached |
| CRM-ADH-SATP-icsB | 58.3 | — | — | 63.0 | 65.6 | — | — | 81.4 |
| | | | | => 29 | => 21 | | | => 9 |
| | | | | carboxyls | ADHs | | | carbohydrates |
| | | | | activated | activated | | | attached |
| Haemophilus | | | | | | | | |
| influenzae | | | | | | | | |
| CRM-BMPH-lic1lpsA | 58.3 | — | 65.0 | — | — | — | 87.7 | — |
| | | | => 36 | | | | => 13 | |
| | | | carboxyls | | | | carbohydrates | |
| | | | activated | | | | attached | |
| CRM-ADH-SATP- | 58.3 | — | — | nd | 66.9 | — | — | 108.0 |
| lic1lpsA | | | | | => 29 | | | => 19 |
| | | | | | ADHs | | | carbohydrates |
| | | | | | activated | | | attached |
| Mannheimia | | | | | | | | |
| haemolytica | | | | | | | | |
| CRM-ADH-SATP-losB | 58.3 | — | — | 62.7 | 65.5 | — | — | 84.9 |
| | | | | => 27 | => 21 | | | => 10 |
| | | | | carboxyls | ADHs | | | carbohydrates |
| | | | | activated | activated | | | attached |
| Moraxella catarrhalis | | | | | | | | |
| CRM-BMPH-lgt2 | 58.3 | — | 63.0 | — | — | — | 87.9 | — |
| | | | => 24 | | | | => 11 | |
| | | | carboxyls | | | | carbohydrates | |
| | | | activated | | | | attached | |
| CRM-ADH-SATP-lgt2 | 58.3 | — | — | 61.7 | 64.8 | — | — | 101.2 |
| lgt4 | | | | => 21 | => 21 | | | => 18 |
| (Dicty) | | | | carboxyls | ADHs | | | carbohydrates |
| | | | | activated | activated | | | attached |

TABLE 2
Bactericidal titers¹ of pre- and post-immunisation rabbit sera
against Neisseria meningitidis stains MC58 and H44/76 wt and lgtA
mutants. Baby rabbit serum was used as the source of complement.

| Sera/Strain | Pre/Post | MC58 | H44/76 | MC58 lgtA | H44/76 lgtA |
|---|---|---|---|---|---|
| BMPH | | | | | |
| conjugate | | | | | |
| RV1 | Pre | 32 | 16 | 32 | <8 |
| | Post | 8 | 32 | 16 | <8 |
| RV2 | Pre | 4 | 8 | 32 | <8 |
| | Post | 4 | 16 | 256² | 128 |
| RV3 | Pre | 4 | 16 | 4 | <8 |
| | Post | 8 | 16 | 128 | 128 |
| RV4 | Pre | 4 | 16 | 32 | <8 |
| | Post | 8 | 16 | 32 | <8 |
| RV5 | Pre | 4 | 16 | <4 | <8 |
| | Post | 4 | 8 | 64 | 64 |
| RV6 | Pre | <4 | 16 | <4 | <8 |
| | Post | 4 | 16 | >2048³ | >4096 |
| RC7 | Pre | 4 | 16 | 4 | 8 |
| | Post | 8 | 32 | 4 | <8 |
| RC8 | Pre | 4 | 16 | 4 | 16 |
| | Post | 8 | 16 | 8 | <8 |
| ADH-SATP | | | | | |
| conjugate | | | | | |
| RLAV1 | Pre | 16 | 32 | 4 | 4 |
| | Post | 16 | 256 | 1024 | 1024 |
| RLAV2 | Pre | 16 | 64 | 4 | 16 |
| | Post | 16 | 1024 | >2048 | >2048 |
| RLAV3 | Pre | 16 | 32 | 4 | 4 |
| | Post | <16 | 128 | 1024 | 2048 |
| RLAC4 | Pre | 16 | 64 | 4 | 4 |
| | Post | 32 | 512 | 4 | 8 |
| RLAC5 | Pre | 32 | 64 | 64 | 4 |
| | Post | 4 | 256 | 4 | 4 |
| +ve control | | 2048 | 4096 | 4096 | 4096 |

¹Bactericidal titers expressed as the reciprocal of the serum dilution yielding >=50% bactericidal killing.
²Titers in bold indicate hint of killing
³Titers in bold and underlined illustrate bactericidal killing.

TABLE 3
Bactericidal titers¹ of pre- (D0) and post- (D70) immunisation
rabbit sera against Moraxella catarrhalis stains serotype A wt and lgt2
mutant. Baby rabbit complement (⅛ dilution) was used.

| Sera/Strain | Pre/Post | Mc wt | Mc lgt2 |
|---|---|---|---|
| Rabbit | | | |
| RMCV1 | Pre | <5 | <5 |
| | Post | <5 | >10² |
| RMCV2 | Pre | <5 | <5 |
| | Post | <5 | >25³ |
| RMCV3 | Pre | <5 | <5 |
| | Post | <5 | >25 |
| +ve control⁴ | | nd | 100 |

¹Bactericidal titers expressed as the reciprocal of the serum dilution yielding >=50% bactericidal killing when compared to the no antibody control.
²Titers in bold indicate hint of killing
³Titers in bold and underlined illustrate bactericidal killing.
⁴Positive control was mouse monoclonal antibody MC2-1

TABLE 4
Bactericidal titers¹ of pre- (D0) and post- (D70) immunisation rabbit
sera against Moraxella catarrhalis stains serotype A wt and lgt2/lgt4
mutant. Baby rabbit complement (⅛ dilution) was used.

| Sera/Strain | Pre/Post | Mc wt | Mc lgt2/lgt4 |
|---|---|---|---|
| Rabbit | | | |
| RMDV3 | Pre | <5 | <5 |
| | Post | >10 | 50² |
| RMDV5 | Pre | <5 | <5 |
| | Post | >10 | 50 |
| RMDV6 | Pre | nd | <5 |
| | Post | nd | 25 |
| +ve control³ | | nd | >10 |

¹Bactericidal titers expressed as the reciprocal of the serum dilution yielding >=50% bactericidal killing when compared to the no antibody control.
²Titers in bold and underlined illustrate bactericidal killing.
³Positive control was mouse monoclonal antibody MC2-1

TABLE 5
Bactericidal titers¹ of pre- (D0) and post-immunisation (D70) rabbit
sera against Neisseria meningitidis stains MC58 and H44/76 wt and MC58
galE mutant. Baby rabbit serum was used as the source of complement.

| Sera/Strain | Pre/Post | MC58 | H44/76 | MC58 galE |
|---|---|---|---|---|
| RGV1 | Pre | 42 | 16 | 4 |
| | Post | 4 | 32 | 256³ |
| RGV4 | Pre | 4 | 32 | 4 |
| | Post | 4 | 64 | 128 |
| RGV6 | Pre | 4 | 16 | 32 |
| | Post | 16² | 64 | 128 |

¹Bactericidal titers expressed as the reciprocal of the serum dilution yielding >=50% bactericidal killing.
²Titers in bold indicate hint of killing
³Titers in bold and underlined illustrate bactericidal killing.

TABLE 6
Bactericidal titers¹ of pre- (D0) and post-immunisation (D42) rabbit
sera against Neisseria meningitidis stains MC58 and H44/76 wt and
MC58 lgtB mutant. Baby rabbit serum was used as the source of
complement.

| Sera/Strain | Pre/Post | MC58 | H44/76 | MC58 lgtB |
|---|---|---|---|---|
| RBV1 | Pre | 4 | 32 | Tox³ |
| | Post | 4 | 32 | Tox |
| RBV2 | Pre | 4 | 32 | Tox |
| | Post | 4 | 64 | Tox |
| RBV3 | Pre | 4 | 32 | Tox |
| | Post | 4 | 512² | Tox |
| RBV4 | Pre | 4 | 64 | Tox |
| | Post | 8 | 128 | Tox |
| RBV5 | Pre | 4 | 16 | Tox |
| | Post | 4 | 16 | Tox |

¹Bactericidal titers expressed as the reciprocal of the serum dilution yielding >=50% bactericidal killing.
²Titers in bold and underlined illustrate bactericidal killing.
³Complement alone killed lgtB strains

TABLE 7
Bactericidal titers¹ of pre- (D0) and post-immunisation (D42) rabbit
sera against Neisseria meningitidis stains MC58 and H44/76 wt and MC58
icsB mutant. Baby rabbit serum was used as the source of complement.

| Sera/Strain | Pre/Post | MC58 | H44/76 | MC58 icsB |
|---|---|---|---|---|
| RNIV2 | Pre | 4 | 16 | 8 |
| | Post | 8 | 128² | >2048 |

¹Bactericidal titers expressed as the reciprocal of the serum dilution yielding >=50% bactericidal killing.
²Titers in bold and underlined illustrate bactericidal killing.

1. An immunogenic conjugate for eliciting a specific immune response to a Gram negative bacterium having a lipopolysaccharide (LPS) endotoxin, said conjugate having the formula I: 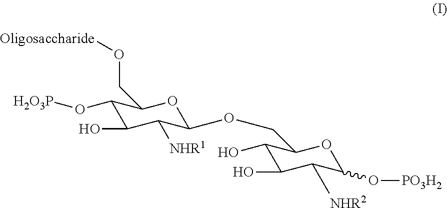
wherein one of R¹ and R² is a linker that is attached to a carrier protein, and the other of R¹ and R² is H or a C1-C20 acyl group;
‘Oligosaccharide’ represents at least five saccharide rings wherein each oligosaccharide comprises a saccharide having a general formula of: 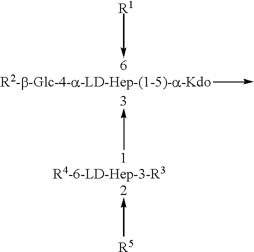
wherein R¹ is H or α-D-glucose;
R² is H, β-D-glucose, β-D-galactose or a disaccharide of β-N-acetyl-D-glucosamine linked to the 3-position of a β-D-galactose, α-DD-heptose or α-LD-heptose;
R³ is H, phosphoethanolamine or α-D-glucose;
R4 is H or phosphoethanolamine; and
R5 is α-N-acetyl-D-glucosamine, α-LD-heptose or a disaccharide of or β-D-Glc-2-α-LD-Hep or a trisaccharide of β-D-Gal-(1-4)-β-D-Glc-2-α-LD-Hep or a tetrasaccharide of α-D-Gal-(1-4)-β-D-Gal-(1-4)-β-D-Glc-2-α-LD-Hep or a pentasaccharide of β-D-GalNAc-(1-3)-α-D-Gal-(1-4)-β-D-Gal-(1-4)-β-D-Glc-2-α-LD-Hep.
and wherein the conjugate retains each phosphate and each phosphoethanolamine present in the corresponding portions of the natural LPS of the Gram negative bacterium;
or a pharmaceutically acceptable salt thereof.

2. The immunogenic conjugate according to claim 1 wherein the Gram negative bacterium is Neisseria meningitidis, and R1 is H; R2 is H, β-D-glucose, β-D-galactose or a disaccharide of β-N-acetyl-D-glucosamine linked to the 3-position of a β-D-galactose; R3 is H, phosphoethanolamine or α-D-glucose; R4 is H or phosphoethanolamine and R5 is α-N-acetyl-D-glucosamine.

3. The immunogenic conjugate according to claim 1 wherein the Gram negative bacterium is Haemophilus influenzae; R1 is H; R2 is H; R3 is H; R4 is phosphoethanolamine; and R5 is α-LD-heptose or a disaccharide of or β-D-Glc-2-α-LD-Hep or a trisaccharide of β-D-Gal-(1-4)-β-D-Glc-2-α-LD-Hep or a tetrasaccharide of α-D-Gal-(1-4)-β-D-Gal-(1-4)-β-D-Glc-2-α-LD-Hep or a pentasaccharide of β-D-GalNAc-(1-3)-α-D-Gal-(1-4)-β-D-Gal-(1-4)-β-D-Glc-2-α-LD-Hep.

4. The immunogenic conjugate according to claim 1 wherein the Gram negative bacterium is For Mannheimia haemolytica, R1 is α-D-glucose; R2 is α-DD-heptose; R3 is H; R4 is H; and R5 is α-LD-heptose.

5. The immunogenic conjugate according to claim 1 wherein the Gram negative bacterium is Actinobacillus pleuropneumoniae, R1 is α-D-glucose; R2 is α-DD-heptose; R3 is H; R4 is H; and R5 is α-LD-heptose.

6. The immunogenic conjugate according to claim 1 wherein the Gram negative bacterium is Pasteurella multocida; R1 is α-D-glucose; R2 is H or α-LD-heptose; R³ is H or phosphoethanolamine; R⁴ is H and R⁵ is α-LD-heptose.

7. The immunogenic conjugate of claim 1, wherein the oligosaccharide is selected from the group consisting of Formula IIa, Formula IIb, Formula IIc, Formula IId, Formula IIe and Formula IIf 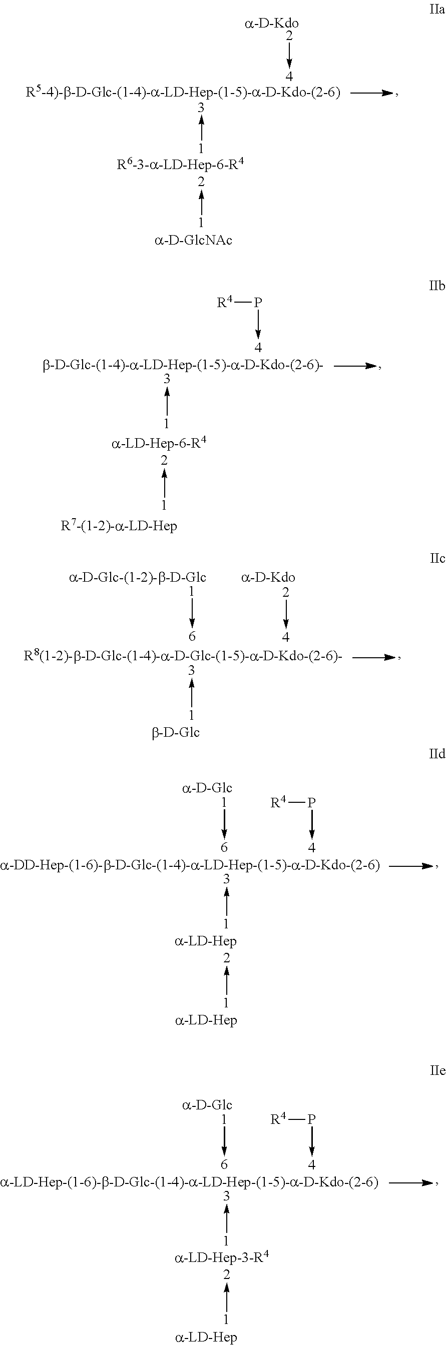 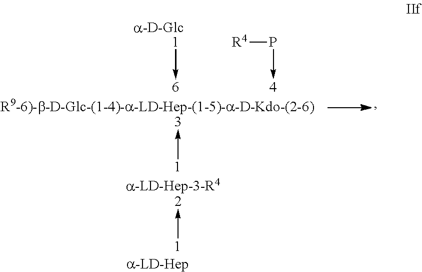
and
wherein P is —PO₃H₂;
each→indicates a point of attachment for the Oligosaccharide in Formula I;
R⁴ is independently at each occurrence H or PEtn;
PEtn is phosphoethanolamine, P . . . PEtn is ethanolamine pyrophosphate, and PCho is phosphorylcholine;
R⁵ is H, beta-D-glucose, beta-D-galactose or disaccharide of beta-N-acetyl-D-glucosamine;
R⁶ is H, phosphoethanolamine (PEtn) or alpha-D-glucose;
R⁷ is selected from the group consisting of H, beta-D-Glc, a disaccharide of beta-D-Gal-(1-4)-beta-D-Glc, a trisaccharide of alpha-D-Gal-(1-4)-beta-D-Gal-(1-4)-beta-D-Glc and a tetrasaccharide of beta-D-GalNAc-(1-3)-alpha-D-Gal-(1-4)-beta-D-Gal-(1-4)-beta-D-Glc;
R⁸ is H, alpha-N-acetyl-D-glucosamine or alpha-D-glucose; and
R⁹ is H or alpha-LD heptose.

8. The immunogenic conjugate of claim 1, wherein the linker is a group that connects the carbohydrate and the carrier protein portions of the conjugate, and comprises 2-40 atoms selected from the group consisting of C, S, O and N.

9. (canceled)

10. The immunogenic conjugate of claim 8, wherein the linker consists of one or more groups selected from L or D amino acids, alkylene, alkenylene, alkynylene, heteroalkylene, heteroalkenylene, heteroalkynylene, heterocyclyl, alkyleneheterocyclylalkylene, each of which is optionally substituted with one or more substituents selected from the group consisting of alkyl, ═O, halo, COOR, CONR₂, SO₂NR₂, NRSO₂R, OR, NR₂, and CN; wherein each R is independently H or C1-C4 alkyl.

11. The immunogenic conjugate of claim 1, wherein the linker connects an amine of formula (I) to a carboxylate or amine on the carrier protein, and the linker is a bond between the nitrogen of the glucosamine portion of the LPS and a carbon atom of the carrier protein, or the linker is selected from the group consisting of —(C═O)—X¹—NH—, —(C═O)—X¹—C(═O)—, a peptide linker comprising two or more amino acid moieties, 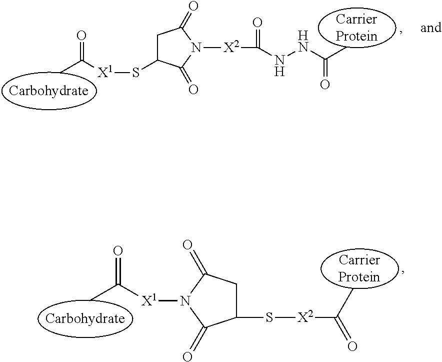
each of which may be optionally substituted,
wherein X¹ and X² are each independently selected from the group consisting of C1-C8 alkylene, C1-C8 alkenylene, C1-C8 alkynylene, C1-C8 heteroalkylene, C1-C8 heteroalkenylene and C1-C8 heteroalkynylene.

12. The immunogenic conjugate of claim 1, wherein R¹ is H or a C1-20 acyl group and R² is a linker that is attached to a carrier protein.

13. The immunogenic conjugate of claim 1, wherein R¹ is a linker that is attached to a carrier protein and R² is H or a C1-20 acyl group.

14. (canceled)

15. The immunogenic conjugate of claim 1, wherein the carrier protein is selected from the group consisting of CRM₁₉₇, tetanus toxoid (TT), human serum albumin (HSA), keyhole limpet hemocyanin (KLH), polydextran and MAP-4 peptide.

16. A compound of Formula III: 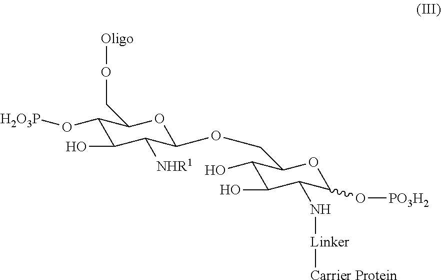
wherein said carrier protein is selected from the group consisting of CRM₁₉₇, tetanus toxoid (TT), human serum albumin (HSA), keyhole limpet hemocyanin (KLH), polydextran and MAP-4 peptide;
Oligo represents an oligosaccharide containing at least five contiguous saccharide rings of a moiety selected from the group consisting of 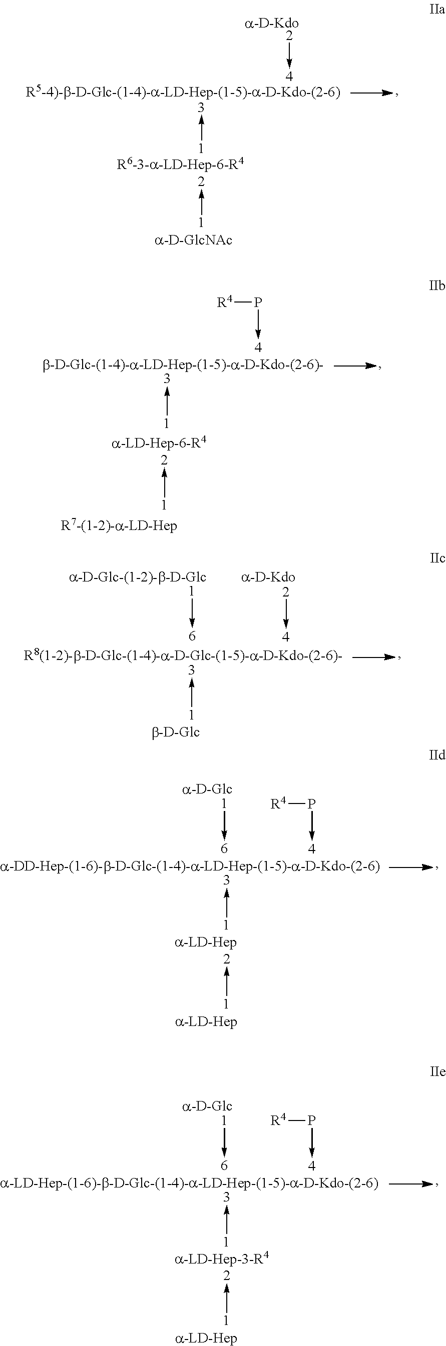 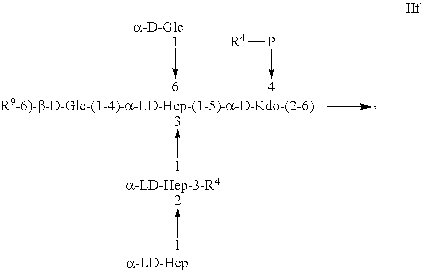
R¹ is H or an acyl group; and
said Linker is selected from the group consisting of a bond between the nitrogen of the glucosamine portion of the LPS and a carbon atom of the carrier protein, (C═O)C1-C8 alkylene, (C═O)C1-C8 alkenylene, (C═O)C1-C8 alkynylene, C1-C8 alkylene(C═O), C1-C8 alkenylene(C═O), C1-C8 alkynylene(C═O), (C═O)C1-C8 heteroalkylene, (C═O)C1-C8 heteroalkenylene, (C═O)C1-C8 heteroalkynylene, a peptide linker, C2-C20 poly(ethylene glycol), 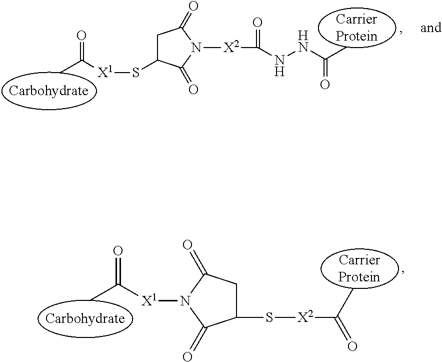
wherein the linker is the group connecting the carbohydrate and the carrier protein portions of the conjugate; and
X¹ and X² are each independently selected from the group consisting of C1-C8 alkylene, C1-C8 alkenylene, C1-C8 alkynylene, C1-C8 heteroalkylene, C1-C8 heteroalkenylene and C1-C8 heteroalkynylene;
or a pharmaceutically acceptable salt thereof.

17. An immunogenic conjugate for eliciting a specific immune response to a Gram negative bacterium having a lipopolysaccharide (LPS) endotoxin, said conjugate having at least 5 molecules of the carbohydrate depicted as 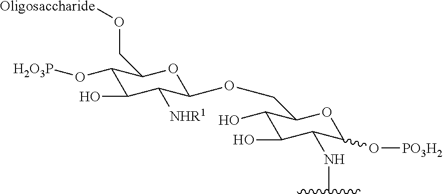
or a pharmaceutically acceptable salt thereof conjugated to a single carrier protein via a linker;
wherein one of R¹ and R² is a linker that is attached to a carrier protein, and the other of R¹ and R² is H or a C1-C20 acyl group;
‘Oligosaccharide’ represents at least five saccharide rings that comprise the corresponding saccharide rings of the lipopolysaccharide endotoxin of the Gram negative bacterium,
and wherein the conjugate retains each phosphate and each phosphoethanolamine present in the corresponding portions of the natural LPS of the Gram negative bacterium;
or a pharmaceutically acceptable salt thereof.

18. The immunogenic conjugate according to claim 17 wherein each saccharide has a general formula of: 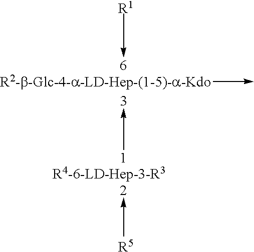
wherein R¹ is H or α-D-glucose;
R² is H, β-D-glucose, β-D-galactose or a disaccharide of β-N-acetyl-D-glucosamine linked to the 3-position of a β-D-galactose, α-DD-heptose or α-LD-heptose;
R³ is H, phosphoethanolamine or α-D-glucose;
R⁴ is H or phosphoethanolamine; and
R⁵ is α-N-acetyl-D-glucosamine, α-LD-heptose or a disaccharide of or β-D-Glc-2-α-LD-Hep or a trisaccharide of β-D-Gal-(1-4)-β-D-Glc-2-α-LD-Hep or a tetrasaccharide of α-D-Gal-(1-4)-β-D-Gal-(1-4)-β-D-Glc-2-α-LD-Hep or a pentasaccharide of β-D-GalNAc-(1-3)-α-D-Gal-(1-4)-β-D-Gal-(1-4)-β-D-Glc-2-α-LD-Hep.

19. An immunogenic conjugate for eliciting a specific immune response to a Gram negative bacterium having a lipopolysaccharide (LPS) endotoxin, said conjugate depicted as 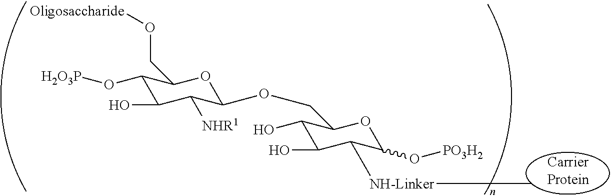
or a pharmaceutically acceptable salt thereof, wherein n is at least 5;
wherein one of R¹ and R² is a linker that is attached to a carrier protein, and the other of R¹ and R² is H or a C1-C20 acyl group;
‘Oligosaccharide’ represents at least five saccharide rings that comprise the corresponding saccharide rings of the lipopolysaccharide endotoxin of the Gram negative bacterium,
and wherein the conjugate retains each phosphate and each phosphoethanolamine present in the corresponding portions of the natural LPS of the Gram negative bacterium;
or a pharmaceutically acceptable salt thereof.

20. The immunogenic conjugate according to claim 19 wherein each saccharide has a general formula of: 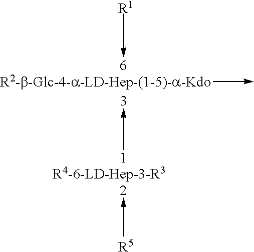
wherein R¹ is H or α-D-glucose;
R² is H, β-D-glucose, β-D-galactose or a disaccharide of β-N-acetyl-D-glucosamine linked to the 3-position of a β-D-galactose, α-DD-heptose or α-LD-heptose;
R³ is H, phosphoethanolamine or α-D-glucose;
R4 is H or phosphoethanolamine; and
R5 is α-N-acetyl-D-glucosamine, α-LD-heptose or a disaccharide of or β-D-Glc-2-α-LD-Hep or a trisaccharide of β-D-Gal-(1-4)-β-D-Glc-2-α-LD-Hep or a tetrasaccharide of α-D-Gal-(1-4)-β-D-Gal-(1-4)-β-D-Glc-2-α-LD-Hep or a pentasaccharide of β-D-GalNAc-(1-3)-α-D-Gal-(1-4)-β-D-Gal-(1-4)-β-D-Glc-2-α-LD-Hep.

21. (canceled)

22. (canceled)

23. (canceled)

24. A method to make an LPS-based immunological conjugate that induces an immune response effective against a Gram negative bacterium, comprising the steps of:
obtaining a lipopolysaccharide (LPS) from the Gram negative bacterium;
removing acyl groups linked to oxygen on the di-glucosamine of the reducing end portion of the LPS;
removing at least one acyl group linked to N of the di-glucosamine reducing end of the LPS to provide an amine group;
protecting any phosphoethanolamine groups attached to the oligosaccharide portion of the LPS:
attaching a first end of a linking group to an amine group on the di-glucosamine;
attaching a second end of the linking group to a carrier moiety; and
de-protecting where necessary the protected phosphoethanolamine groups.

25. The method of claim 24, wherein the LPS-derived moiety comprises a phosphate on the anomeric center of the reducing glucosamine moiety, and the phosphate is retained in the immunogenic conjugate.

26. The method of claim 24, wherein each phosphate and each phosphoethanolamine of the LPS from the Gram negative bacterium is preserved in the immunogenic conjugate.

27. The method of claim 24, wherein removing at least one acyl group attached to N of the reducing di-glucosamine comprises contacting the LPS or modified LPS with an amidase that selectively removes the acyl group.

28. The method of claim 27, wherein the amidase is from Dictyostelium discoideum.

29. The method of claim 24, wherein the step of protecting at least one phosphoethanolamine group comprises acylation of the amine of a phosphoethanolamine group to form a carbamate.

30. The method of claim 29, wherein the carbamate is a methyl carbamate, t-butyl carbamate or benzyl carbamate.

31. The method of claim 24, wherein the Gram negative bacterium is Neisseria meningitidis.

32. (canceled)

33. (canceled)

34. (canceled)

35. (canceled)

36. (canceled)

37. An LPS-based immunological conjugate prepared by the method of claim 24.

38. An immunological composition comprising the conjugate of claim 37 admixed with at least one vaccine adjuvant.

39. (canceled)
